(19) United States
(12) Patent Application Publication
(10) Pub. No.: US 2024/0375024 A1
(43) Pub. Date: Nov. 14, 2024

(54) METHODS OF ISOLATING A COMPOSITION COMPRISING A TARGET MOLECULE

(71) Applicant: DEWPOINT THERAPEUTICS, INC., Boston, MA (US)

(72) Inventors: Danielle Lauren SOOKIASIAN, Cohasset, MA (US); Roberto Neves NUNES, East Bridgewater, MA (US); Peter Jeffrey DANDLIKER, Quincy, MA (US); Benjamin Diamon LEVIN, Needham, MA (US); Juwina WIJAYA, Arlington, MA (US); Kamran Daniel RIZZOLO ROUSTAYAN, Canton, MA (US)

(21) Appl. No.: 18/684,592

(22) PCT Filed: Aug. 19, 2022
(86) PCT No.: PCT/US2022/040890

§ 371 Date: Feb. 16, 2024

Related U.S. Application Data
(60) Provisional application No. 63/235,594, filed on Aug. 20, 2021.

(51) Int. Cl. B01D 15/38

(52) U.S. Cl. CPC B01D 15/3823

(57) ABSTRACT

The present disclosure provides immunoprecipitation (IP) methods such as antibody-mediated techniques using a bridged tandem antibody complex. In other aspects, the present disclosure also provides compositions, kits, and systems useful for use in the immunoprecipitation methods described herein.

### CROSS-REFERENCE TO RELATED APPLICATIONS

This application claims priority benefit of U.S. Provisional Patent Application No. 63/235,594, filed on Aug. 20, 2021, the contents of which are hereby incorporated herein by reference in its entirety.

### TECHNICAL FIELD

The present disclosure is directed to, in certain aspects, methods of isolating a composition comprising a target molecule, e.g., immunoprecipitation (IP) methods such as antibody-mediated techniques using a bridged tandem antibody complex. In other aspects, the present disclosure also provides compositions, kits, and systems useful for use in the methods described herein.

### BACKGROUND

Techniques for the isolation of biological components from a mixture, such as from a cell, are useful for the study of various biological systems. Such methods, including immunoprecipitation, allow for the identification of one or more components of interest, such as a protein, and the assessment of cellular interactions. Certain techniques for the isolation of biological components from a mixture are known, including affinity purification or immunoprecipitation coupled to mass spectrometry (e.g., AP-MS and IP-MS), crosslinking mass spectrometry, and proximity labeling by enzymatic catalysis, and various co-IP techniques. However, these known methods suffer from low yield and/or loss of associated biological components, such as co-binders, and/or the a lack of purity, namely, the presence of certain co-precipitated contaminants that create high background signal and interfere with the detection of low abundant components of interest, or a component associated therewith. Furthermore, known methods are not adaptable to high-throughput experiments.

All references cited herein, including patent applications and publications, are incorporated by reference in their entirety.

### BRIEF SUMMARY

In some aspects, provided is a method of isolating a composition comprising a target molecule from a sample, the method comprising: (a) admixing the sample with a primary antibody specifically recognizing the target molecule, a secondary antibody comprising an affinity label and specifically recognizing the primary antibody, and a first solid phase comprising a first affinity label complexing agent to form a binding composition, wherein a tandem antibody complex is formed in the binding composition in the presence of the target molecule, wherein the tandem antibody complex comprises the target, the primary antibody, the second antibody, and the first solid phase, wherein the primary antibody binds to the target molecule, wherein the secondary antibody binds to the primary antibody, and wherein the first affinity label complexing agent of the first solid phase forms a complex with the affinity label of the secondary antibody; and (b) isolating the composition comprising the target molecule, wherein isolating the composition comprising the target molecule does not comprise formation of a second tandem antibody complex comprising the primary antibody, the secondary antibody, and a solid phase.

In some embodiments, the sample is a pre-cleared sample, and the method further comprises obtaining the pre-cleared sample by admixing the sample with first clearing agent and a second clearing agent prior to step (a) to form the pre-cleared sample, wherein the first clearing agent comprises a second solid phase and an antibody binding agent that binds to a population of antibodies, and wherein the population of antibodies comprises the primary antibody specifically recognizing the target molecule, wherein the second clearing agent comprises a third solid phase and a second affinity label complexing agent that forms a complex with the affinity label in a free state.

In some embodiments, the isolating step comprises a target molecule pull-down step, wherein the pull-down step comprises isolating the first solid phase, and components associated therewith, from other components in the sample or the pre-cleared sample, wherein the method does not comprise a second target molecule pull-down step. In some embodiments, isolating the first solid phase, and components associated therewith, comprises subjecting the admixed sample to a force to pellet the first solid phase, and components associated therewith. In some embodiments, the isolation further comprises a step of removing the target molecule from the first solid phase.

In other aspects, provided is a method of isolating a composition comprising a target molecule from a sample, the method comprising: (a) admixing a pre-cleared sample with a primary antibody specifically recognizing the target molecule, a secondary antibody comprising an affinity label and specifically recognizing the primary antibody, and a first solid phase comprising a first affinity label complexing agent to form a binding composition, wherein the pre-cleared sample is derived from the sample cleared by a first clearing agent and a second clearing agent, wherein the first clearing agent comprises a second solid phase and an antibody binding agent that binds to a population of antibodies, and wherein the primary antibody belongs to the population of antibodies bound by the antibody binding agent, wherein the second clearing agent comprises a third solid phase and a second affinity label complexing agent that forms a complex with the affinity label, wherein a tandem antibody complex is formed in the binding composition in the presence of the target molecule, wherein the tandem antibody complex comprises the target molecule, the primary antibody, the second antibody, and the first solid phase, wherein the primary antibody binds to the target molecule, wherein the secondary antibody binds to the primary antibody, and wherein the first affinity label complexing agent of the first solid phase forms a complex with the affinity label of the secondary antibody; and (b) isolating the composition comprising the target molecule.

In some embodiments, isolating the composition comprising the target molecule from the pre-cleared sample does not comprise formation of a second tandem antibody complex comprising the primary antibody, the secondary antibody, and another solid phase. In some embodiments, the method further comprises obtaining the pre-cleared sample, wherein obtaining the pre-cleared sample comprises admixing the sample with the first clearing agent comprising the antibody binding agent that binds to the population of antibodies, and the second clearing agent comprising the second affinity label complexing agent that forms a complex with the affinity label. In some embodiments, admixing the sample with first clearing agent and admixing the sample with second clearing agent occurs simultaneously, sequentially, or concurrently.

In some embodiments, the population of antibodies bound by the first clearing agent is characterized by the organism of origin. In some embodiments, the population of antibodies bound by the first clearing agent originate from a human, rabbit, mouse, or pig.

In some embodiments, the population of antibodies bound by the first clearing agent is characterized by the antibody isotype. In some embodiments, the antibody isotope of the population of antibodies bound by the first clearing agent is selected from the group consisting of IgG, IgM, IgD, IgE, and IgA. In some embodiments, the IgG is selected from the group consisting of IgG1, IgG2, IgG3, and IgG4. In some embodiments, the IgA is selected from the group consisting of IgA1 and IgA2.

In some embodiments, the antibody binding agent of the first clearing agent comprises protein G, protein A, protein A/G, protein L, or jacalin.

In some embodiments, the second affinity label complexing agent of the second clearing agent comprises streptavidin, an anti-biotin antibody, or NeutrAvidin.

In some embodiments, the method further comprises separating the first clearing agent and the second clearing agent from the sample to obtain the pre-cleared sample. In some embodiments, separating the first clearing agent and the second clearing agent from the sample occurs after a period of incubation. In some embodiments, the separating the first clearing agent and/or the second clearing agent comprises pelleting the first clearing agent and/or the second clearing agent. In some embodiments, the pelleting comprising centrifugation or magnetic separation.

In some embodiments, the primary antibody is characterized by the antibody isotype. In some embodiments, the antibody isotope of the primary antibody is selected from the group consisting of IgG, IgM, IgD, IgE, and IgA. In some embodiments, the IgG is selected from the group consisting of IgG1, IgG2, IgG3, and IgG4. In some embodiments, the IgA is selected from the group consisting of IgA1 and IgA2.

In some embodiments, the secondary antibody binds to the Fc region of the primary antibody.

In some embodiments, the affinity label of the secondary antibody comprises a ligand, and wherein the first affinity label complexing agent of the first solid phase comprises a receptor for the ligand. In some embodiments, the affinity label of the secondary antibody comprises biotin, and wherein the first affinity label complexing agent of the first solid phase comprises streptavidin. In some embodiments, the affinity label of the secondary antibody comprises biotin, and wherein the first affinity label complexing agent of the first solid phase comprises anti-biotin.

In some embodiments, the affinity label and the second affinity label complexing agent are each portions of a reactive group. In some embodiments, the affinity label of the secondary antibody forms a complex with the first affinity label complexing agent of the first solid phase via click chemistry.

In some embodiments, the first solid phase comprises a bead. In some embodiments, the bead of the first solid phase is a non-magnetic bead configured in a chromatography column.

In some embodiments, the second solid phase and the third solid phase are the same. In some embodiments, the second solid phase and the third solid phase are different.

In some embodiments, the first affinity label complexing agent and the second affinity label complexing agent are the same. In some embodiments, the first affinity label complexing agent and the second affinity label complexing agent are different.

In some embodiments, the method further comprises a washing step. In some embodiments, the washing step comprises: pelleting the binding composition, removing the resulting supernatant, adding the washing solution, pelleting a resulting solution, and removing a resulting solution. In some embodiments, the washing step is repeated at least once. In some embodiments, the washing step is repeated three times.

In some embodiments, pelleting the first solid phase concentrated the compositions comprising the target molecule. In some embodiments, the pelleting comprises centrifugation or magnetic separation.

In some embodiments, the method further comprises eluting the target molecule from the first solid phase using an elution solution. In some embodiments, the elution solution comprises biotin. In some embodiments, the concentration of biotin in the elution solution is at least about 2 nM. In some embodiments, the target molecule is obtained with about 75-95% recovery after eluting.

In some embodiments, the target molecule is a complex, a polypeptide, a polypeptide complex, or a mixed complex.

In some embodiments, the sample is a cell lysate. In some embodiments, the method further comprises lysing a cell to obtain the sample.

In some embodiments, the purity and/or recovery of the target molecule is assessed by SDS-PAGE, immunoblot, mass spectrometry, or liquid-chromatography/mass spectrometry.

In other aspects, provided is a method of isolating a composition comprising a target molecule from a sample, the method comprising: (a) admixing the sample with a first clearing agent and second clearing agent to form a pre-cleared sample, wherein the first clearing agent comprises a second solid phase and an antibody binding agent that binds an antibody species, wherein the antibody species bound by the antibody binding agent is the same as the species of the primary antibody, and wherein the second clearing agent comprises a third solid phase and a second affinity label binding agent that binds the affinity label; (b) admixing the pre-cleared sample with a primary antibody, a secondary antibody, and a first solid phase comprising a first affinity label binding agent, to form a binding composition, wherein a tandem antibody complex is formed in the binding composition in the presence of the target molecule, wherein the tandem antibody complex comprises the target molecule, the primary antibody, the second antibody, and the first solid phase, wherein the primary antibody binds to the target molecule, wherein the secondary antibody binds to the primary antibody, and wherein the first affinity label complexing agent of the first solid phase binds to the affinity label of the secondary antibody; (c) washing the binding composition comprising the tandem antibody complex with a washing solution; and, (d) eluting the composition comprising the target molecule isolated from the tandem antibody complex, to obtain the composition comprising the target molecule isolated from the sample.

In some embodiments, isolating the composition comprising the target molecule from the sample does not comprise use of a second solid phase, wherein the second solid phase binds with the primary antibody, or a portion thereof.

In other aspects, provided is a composition comprising a target molecule obtained from any one of the composition isolation methods described herein.

In some embodiments, the target molecule is a component of a condensate.

In some embodiments, the method further comprises crosslinking the sample prior to performing purification steps. In some embodiments, the crosslinked sample is denatured prior to performing purification steps. In some embodiments, the denaturing comprises heat or chemical denaturation.

In other aspects, provided herein is a kit, comprising a first clearing agent, a second clearing agent, a primary antibody, a secondary antibody, and a first, second, and third solid phase, wherein: the first clearing agent comprises the second solid phase and an antibody binding agent that binds an antibody species, and wherein the antibody species bound by the antibody binding agent is the same as the species of the primary antibody, the second clearing agent comprises the third solid phase and a second affinity label binding agent that binds the affinity label, the primary antibody binds to a target molecule in a sample, the secondary antibody binds to the primary antibody, and the secondary antibody comprises an affinity label, the first solid phase comprises a first affinity label complexing agent that binds to the affinity label of the secondary antibody, forming a binding composition comprising a tandem antibody complex in the presence of the target molecule.

In some embodiments, the kit further comprises reagents for preparing the pre-cleared sample, washing the tandem antibody complex, and eluting the composition comprising the target molecule isolated from the sample. In some embodiments of the kit, the composition comprising the target molecule is isolated from the sample.

### BRIEF DESCRIPTION OF THE DRAWINGS

FIG. 1 shows an exemplary schematic of an antibody mediated co-immunoprecipitation method described herein.

FIG. 2 shows an exemplary workflow of an antibody mediated co-immunoprecipitation method described herein.

FIG. 3 shows an SDS-PAGE gel followed by an immunoblot against Protein 1 and Protein 2 in HeLa wild type cells, illustrating ˜80-90% recovery of target proteins (Proteins 1 and 2) following application of a co-immunoprecipitation method described herein.

FIG. 4 shows an SDS-PAGE gel followed by an immunoblot against six different proteins (Proteins A-F) in four different cell lines (AC16 mutant (Mut), H9c2 wildtype (WT) WT, H9c2 mutant (Mut), and HeLa wild type (WT)), following application of a co-immunoprecipitation method described herein.

FIG. 5A shows a histogram of intensity values of a bait protein as measured using mass spectrometry for capture methodology using different concentration of the DSP crosslinker. FIG. 5B shows the number of known condensate proteins identified in the top 10% of mass spectrometry identified proteins by abundance.

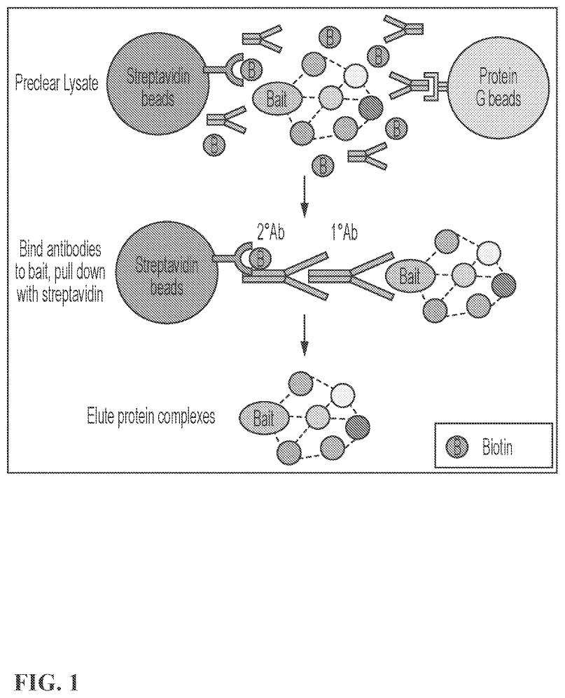

### DETAILED DESCRIPTION

The present application provides, in some aspects, methods of isolating a composition comprising a target molecule from a sample. The present disclosure is based, at least in part, on the inventors' unexpected findings that an isolation technique involving a one-step pull-down using the formation of a tandem antibody complex greatly improves the yield and purity of a target molecule, and in certain aspects biologically relevant co-binders thereof, isolated from a sample as compared to immunoprecipitation techniques known in the art. As described in some aspects herein, certain pre-clearing steps, such as a two-part pre-clearing step based on the agents used to form the tandem antibody complex, are performed in conjunction with the one-step pull-down to prepare the sample for application of certain steps of the improved isolation technique. The use of a pre-clearing step(s) further improves isolation methods taught herein by removing competing nonspecific binders prior to the formation of a tandem antibody complex configured to capture a target molecule of interest. The improved yields of a target molecule obtained with the provided isolation methods are particularly useful for capturing biological relevant co-binders with the target molecule (e.g., components associated with the target molecule in a biological context) at amounts that enable detection. In addition to achieving a high yield and purity of an isolated target molecule, and biologically relevant co-binders, the isolation methods described herein have a streamlined workflow and can be completed in a rapid manner. For example, the methods provided herein do not require a second target molecule pull-down step (e.g., does not require formation of a second tandem antibody complex), and thus are quicker and require less sample processing than such sequential pull down techniques. The isolation techniques taught herein enable improvements in the study of target molecules, and biologically relevant co-binders, in biological systems, and are especially useful for the study of low abundant target molecule and/or biologically relevant co-binder species. For example, the isolation techniques taught herein enable the study of biological condensate and components thereof.

Thus, in some aspects, provided herein is a method of isolating a composition comprising a target molecule from a sample, the method comprising admixing the sample with a primary antibody specifically recognizing the target molecule, a secondary antibody comprising an affinity label and specifically recognizing the primary antibody, and a first solid phase comprising a first affinity label complexing agent to form a binding composition. In some embodiments, a tandem antibody complex is formed in the binding composition in the presence of the target molecule, wherein the tandem antibody complex comprises the target, the primary antibody, the second antibody, and the first solid phase, wherein the primary antibody binds to the target molecule, wherein the secondary antibody binds to the primary antibody, and wherein the first affinity label complexing agent of the first solid phase forms a complex with the affinity label of the secondary antibody. In some embodiments, the composition comprising the target molecule is isolated from the sample (e.g., by washing and eluting), wherein isolating the composition comprising the target molecule does not comprise formation of a second tandem antibody complex comprising the primary antibody, the secondary antibody, and a solid phase.

In other aspects, provided herein is a method of isolating a composition comprising a target molecule from a sample, the method comprising admixing a pre-cleared sample with a primary antibody specifically recognizing the target molecule, a secondary antibody comprising an affinity label and specifically recognizing the primary antibody, and a first solid phase comprising a first affinity label complexing agent to form a binding composition. In some embodiments, the pre-cleared sample is derived from the sample cleared by a first clearing agent and a second clearing agent, wherein the first clearing agent comprises a second solid phase and an antibody binding agent that binds to a population of antibodies, and wherein the primary antibody belongs to the population of antibodies bound by the antibody binding agent, wherein the second clearing agent comprises a third solid phase and a second affinity label complexing agent that forms a complex with the affinity label. In some embodiments, a tandem antibody complex is formed in the binding composition in the presence of the target molecule, wherein the tandem antibody complex comprises the target molecule, the primary antibody, the second antibody, and the first solid phase, wherein the primary antibody binds to the target molecule, wherein the secondary antibody binds to the primary antibody, and wherein the first affinity label complexing agent of the first solid phase forms a complex with the affinity label of the secondary antibody. In some embodiments, the composition comprising the target molecule is isolated from the sample (e.g., by washing and eluting).

In other aspects, provided herein is a method of isolating a composition comprising a target molecule from a sample, the method comprising admixing the sample with a first clearing agent and second clearing agent to form a pre-cleared sample, wherein the first clearing agent comprises a second solid phase and an antibody binding agent that binds an antibody species, wherein the antibody species bound by the antibody binding agent is the same as the species of the primary antibody, and wherein the second clearing agent comprises a third solid phase and a second affinity label binding agent that binds the affinity label. In some embodiments, the method further comprises admixing the pre-cleared sample with a primary antibody, a secondary antibody, and a first solid phase comprising a first affinity label binding agent, to form a binding composition, wherein a tandem antibody complex is formed in the binding composition in the presence of the target molecule, wherein the tandem antibody complex comprises the target molecule, the primary antibody, the second antibody, and the first solid phase, wherein the primary antibody binds to the target molecule, wherein the secondary antibody binds to the primary antibody, and wherein the first affinity label complexing agent of the first solid phase binds to the affinity label of the secondary antibody. In some embodiments, the method further comprises washing the binding composition comprising the tandem antibody complex with a washing solution. In some embodiments, the method further comprises eluting the composition comprising the target molecule isolated from the tandem antibody complex, to obtain the composition comprising the target molecule isolated from the sample.

In other aspects, provided herein is a composition comprising a target molecule obtained from any of the composition isolation methods described.

In other aspects, provided herein is a kit, comprising a first clearing agent, a second clearing agent, a primary antibody, a secondary antibody, and a first, second, and third solid phase. In some embodiments, the first clearing agent comprises the second solid phase and an antibody binding agent that binds an antibody species, and wherein the antibody species bound by the antibody binding agent is the same as the species of the primary antibody. In some embodiments, the second clearing agent comprises the third solid phase and a second affinity label binding agent that binds the affinity label. In some embodiments, the primary antibody binds to a target molecule in a sample. In some embodiments, the secondary antibody binds to the primary antibody, and the secondary antibody comprises an affinity label. In some embodiments, the first solid phase comprises a first affinity label complexing agent that binds to the affinity label of the secondary antibody, forming a binding composition comprising a tandem antibody complex in the presence of the target molecule.

In some embodiments, the sample contains, or is suspected of containing, a condensate. In some embodiments, the condensate is a cellular condensate. In some embodiments, the target molecule is, or can be, associated with a condensate, such as a cellular condensate.

### I. Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-translational modifications of the polypeptide, for example, glycosylation, sialylation, acetylation, phosphorylation, and the like.

The term “antibody” includes full-length antibodies and antigen-binding fragments thereof. A full-length antibody comprises two heavy chains and two light chains. The variable regions of the light and heavy chains are responsible for antigen binding. The variable region in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as lgG1 (γ1 heavy chain), lgG2 (γ2 heavy chain), lgG3 (γ3 heavy chain), lgG4 (γ4 heavy chain), lgA1 (α1 heavy chain), or lgA2 (α2 heavy chain).

The term “antigen-binding fragment” as used herein refers to an antibody fragment including, for example, a diabody, a Fab, a Fab′, a F (ab′) 2, an Fv fragment, a disulfide stabilized Fv fragment (dsFv), a (dsFv)2, a bispecific dsFv (dsFv-dsFv′), a disulfide stabilized diabody (ds diabody), a single-chain antibody molecule (scFv), an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

The term “epitope” as used herein refers to the specific group of atoms or amino acids on an antigen to which an antibody binds. Two antibodies may bind the same epitope within an antigen if they exhibit competitive binding for the antigen.

As use herein, the term “specifically binds” or “specific for” refers to measurable and reproducible interactions, such as binding between a target and an antibody that is determinative of the presence of the target in the presence of a heterogeneous population of molecules, including biological molecules. For example, an antibody that specifically binds to a target (which can be an epitope) is an antibody that binds this target with greater affinity, avidity, more readily, and/or with greater duration than its bindings to other targets. In some embodiments, an antibody that specifically binds to an antigen reacts with one or more antigenic determinants of the antigen with a binding affinity that is at least about 10 times its binding affinity for other targets.

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other.

The terms “Fc receptor” or “FcR” are used to describe a receptor that binds to the Fc region of an antibody. In some embodiments, an FcR of this invention is one that binds an IgG antibody (a γ receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see review M. in Daëron, Annu. Rev. Immunol. 15:203-234 (1997)). The term includes allotypes, such as FcγRIIIA allotypes: FcγRIIIA-Phe158, FcγRIIIA-Val158, FcγRIIA-R131 and/or FcγRIIA-H131. FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

A “functional Fc fragment” possesses an “effector function” of a native sequence Fc region. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g. B cell receptor; BCR), etc. Such effector functions generally require the Fc region to be combined with a binding domain (e.g. an antibody variable domain) and can be assessed using various assays known in the art.

The term “individual” refers to a mammal and includes, but is not limited to, human, bovine, horse, feline, canine, rodent, or primate.

As used herein, “condensate” means a non-membrane-encapsulated compartment formed by phase separation of one or more of proteins and/or other macromolecules (including all stages of phase separation).

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

It will also be understood by those skilled in the art that changes in the form and details of the subject matter described herein may be made without departing from the scope of this disclosure. In addition, although various advantages, aspects, and objects have been described with reference to various implementations, the scope of this disclosure should not be limited by reference to such advantages, aspects, and objects. The scope of this disclosure should be determined with, e.g., reference to the appended claims.

### II. Methods Directed to Isolating a Composition Comprising a Target from a Sample

The present disclosure provides methods for isolating a composition comprising a target from a sample. In some embodiments, isolating encompasses obtaining a desired level of purity of the target in the composition comprising the target.

In some aspects, the method comprises one or more steps that result in the formation of a tandem bridge complex in the presence of the target molecule. In some embodiments, the tandem bridge complex is a tandem antibody complex. In some embodiments, the tandem antibody complex is formed by admixing the sample with a primary antibody specifically recognizing the target molecule, a secondary antibody comprising an affinity label and specifically recognizing the primary antibody, and a first solid phase comprising a first affinity label complexing agent to form a binding composition. In some embodiments, the tandem antibody complex is formed when the primary antibody binds to the target molecule, the secondary antibody binds to the primary antibody, and the first affinity label complexing agent of the first solid phase forms a complex with the affinity label of the secondary antibody. In some embodiments, the method comprises admixing the sample with a primary antibody specifically recognizing a target molecule, a secondary antibody comprising an affinity label and specifically recognizing the primary antibody, and a first solid phase comprising a first affinity label complexing agent to form a binding composition.

In some embodiments, the method comprises: (a) admixing the sample with a primary antibody specifically recognizing the target molecule, a secondary antibody comprising an affinity label and specifically recognizing the primary antibody, and a first solid phase comprising a first affinity label complexing agent to form a binding composition, wherein a tandem antibody complex is formed in the binding composition in the presence of the target molecule, wherein the tandem antibody complex comprises the target, the primary antibody, the second antibody, and the first solid phase, wherein the primary antibody binds to the target molecule, wherein the secondary antibody binds to the primary antibody, and wherein the first affinity label complexing agent of the first solid phase forms a complex with the affinity label of the secondary antibody; and (b) isolating the composition comprising the target molecule, wherein isolating the composition comprising the target molecule does not comprise formation of a second tandem antibody complex comprising the primary antibody, the secondary antibody, and a solid phase.

In some aspects, the method comprises one or more pre-clearing steps. In some embodiments, the sample is a pre-cleared sample. In some embodiments, the method further comprises obtaining the pre-cleared sample by admixing the sample with first clearing agent and a second clearing agent prior to step (a) to form the pre-cleared sample. In some embodiments, the admixing of the sample with first clearing agent and admixing the sample with second clearing agent occurs simultaneously, sequentially, or concurrently. In some embodiments, the first clearing agent comprises a solid phase (e.g., a second solid phase) and an antibody binding agent that binds to a population of antibodies, and wherein the population of antibodies comprises the primary antibody specifically recognizing the target molecule. In some embodiments, the population of antibodies bound by the first clearing agent is characterized by the organism of origin (e.g., originate from a human, rabbit, mouse, or pig). In some embodiments, the population of antibodies bound by the first clearing agent is characterized by the antibody isotype. For example, the antibody isotope of the population of antibodies bound by the first clearing agent is selected from the group consisting of IgG, IgM, IgD, IgE, and IgA. In some embodiments, the antibody binding agent of the first clearing agent comprises protein G, protein A, protein A/G, protein L, or jacalin.

In some embodiments, the method further comprises separating the first clearing agent and the second clearing agent from the sample to obtain the pre-cleared sample. In some embodiments, the separating of the first clearing agent and the second clearing agent from the sample occurs after a period of incubation (e.g., 20 minutes or more). In some embodiments, the separating of the first clearing agent and/or the second clearing agent comprises pelleting the first clearing agent and/or the second clearing agent (e.g., by centrifugation or magnetic separation). In some embodiments, the pre-clearing step is repeated more than once.

In some embodiments, the pre-cleared sample is admixed with a primary antibody specifically recognizing the target molecule, a secondary antibody comprising an affinity label and specifically recognizing the primary antibody, and a first solid phase comprising a first affinity label complexing agent to form a binding composition.

In some embodiments, the method further comprises washing the binding composition comprising the tandem antibody complex to separate the tandem antibody complex from the binding composition. In some embodiments, the washing comprises: pelleting the binding composition, removing the resulting supernatant, adding the washing solution, pelleting a resulting solution, and removing a resulting solution. In some embodiments, the washing is repeated at least once (e.g., three times). In some embodiments, the pelleting of the binding composition (e.g., by centrifugation or magnetic separation) comprises pelleting the first solid phase, and concentrates the compositions comprising the target molecule.

In some embodiments, the method comprises eluting the target molecule from the first solid phase using an elution solution. In some embodiments, the elution solution comprises biotin (e.g., 2 nM biotin). In some embodiments, the elution solution comprises a reducing agent. In some embodiments, the elution solution is compatible with downstream analyses to evaluation target molecule recovery (e.g., assessing the purity and/or recovery of the target molecule by SDS-PAGE, immunoblot, mass spectrometry, or liquid-chromatography/mass spectrometry).

In some embodiments, the sample is crosslinked prior to purification analysis. The crosslinking step can occur at any point during the purification process. In some embodiments, the sample is a lysate. In some embodiments, the method further comprises crosslinking the sample prior to lysis. In some embodiments, crosslinking the sample prior to lysis allows biological interactions with a target molecule to be preserved. In some embodiments, the sample is a crosslinked sample. In some embodiments, the crosslinked sample is lysed. In some embodiments, the crosslinked and lysed sample is analyzed by the provided methods. In some embodiments, the method further comprises denaturing the lysate. In some embodiments, the method further comprises denaturing the crosslinked lysate prior to analysis of the sample. In some embodiments, the denaturing comprises chemical denaturing or heat denaturing. In some embodiments, the lysate is heated for at least about 3 minutes and at a temperature of at least about 70° C. In some embodiments, the heat denaturing is followed by immediate transition to ice, such as to prevent renaturation/refolding.

In some aspects, provided herein is a method of isolating a composition comprising a target molecule from a sample, the method comprising: (a) admixing a pre-cleared sample with a primary antibody specifically recognizing the target molecule, a secondary antibody comprising an affinity label and specifically recognizing the primary antibody, and a first solid phase comprising a first affinity label complexing agent to form a binding composition, wherein the pre-cleared sample is derived from the sample cleared by a first clearing agent and a second clearing agent, wherein the first clearing agent comprises a second solid phase and an antibody binding agent that binds to a population of antibodies, and wherein the primary antibody belongs to the population of antibodies bound by the antibody binding agent, wherein the second clearing agent comprises a third solid phase and a second affinity label complexing agent that forms a complex with the affinity label, wherein a tandem antibody complex is formed in the binding composition in the presence of the target molecule, wherein the tandem antibody complex comprises the target molecule, the primary antibody, the second antibody, and the first solid phase, wherein the primary antibody binds to the target molecule, wherein the secondary antibody binds to the primary antibody, and wherein the first affinity label complexing agent of the first solid phase forms a complex with the affinity label of the secondary antibody; and (b) isolating the composition comprising the target molecule.

In some aspects, provided herein is a method of isolating a composition comprising a target molecule from a sample, the method comprising: (a) admixing the sample with a first clearing agent and second clearing agent to form a pre-cleared sample, wherein the first clearing agent comprises a second solid phase and an antibody binding agent that binds an antibody species, wherein the antibody species bound by the antibody binding agent is the same as the species of the primary antibody, and wherein the second clearing agent comprises a third solid phase and a second affinity label binding agent that binds the affinity label; (b) admixing the pre-cleared sample with a primary antibody, a secondary antibody, and a first solid phase comprising a first affinity label binding agent, to form a binding composition, wherein a tandem antibody complex is formed in the binding composition in the presence of the target molecule, wherein the tandem antibody complex comprises the target molecule, the primary antibody, the second antibody, and the first solid phase, wherein the primary antibody binds to the target molecule, wherein the secondary antibody binds to the primary antibody, and wherein the first affinity label complexing agent of the first solid phase binds to the affinity label of the secondary antibody; (c) washing the binding composition comprising the tandem antibody complex with a washing solution; and, (d) eluting the composition comprising the target molecule isolated from the tandem antibody complex, to obtain the composition comprising the target molecule isolated from the sample.

In some aspects, provided herein is a method of isolating a composition comprising a target molecule from a sample, the method comprising: (a) lysing a sample with a lysis solution; (b) admixing the sample lysate with a first clearing agent and second clearing agent to form a pre-cleared sample lysate, wherein the first clearing agent comprises a second solid phase and an antibody binding agent that binds an antibody species, wherein the antibody species bound by the antibody binding agent is the same as the species of the primary antibody, and wherein the second clearing agent comprises a third solid phase and a second affinity label binding agent that binds the affinity label; (c) admixing the pre-cleared sample lysate with a primary antibody, a secondary antibody, and a first solid phase comprising a first affinity label binding agent, to form a binding composition, wherein a tandem antibody complex is formed in the binding composition in the presence of the target molecule, wherein the tandem antibody complex comprises the target molecule, the primary antibody, the second antibody, and the first solid phase, wherein the primary antibody binds to the target molecule, wherein the secondary antibody binds to the primary antibody, and wherein the first affinity label complexing agent of the first solid phase binds to the affinity label of the secondary antibody; (d) washing the binding composition comprising the tandem antibody complex with a washing solution; and, (e) eluting the composition comprising the target molecule isolated from the tandem antibody complex, to obtain the composition comprising the target molecule isolated from the sample.

In some aspects, provided herein is a method of isolating a composition comprising a target molecule from a sample, the method comprising: (a) crosslinking the sample; (b) lysing the crosslinked sample with a lysis solution; (c) admixing the lysate with a first clearing agent and second clearing agent to form a pre-cleared sample lysate, wherein the first clearing agent comprises a second solid phase and an antibody binding agent that binds an antibody species, wherein the antibody species bound by the antibody binding agent is the same as the species of the primary antibody, and wherein the second clearing agent comprises a third solid phase and a second affinity label binding agent that binds the affinity label; (d) admixing the pre-cleared sample lysate with a primary antibody, a secondary antibody, and a first solid phase comprising a first affinity label binding agent, to form a binding composition, wherein a tandem antibody complex is formed in the binding composition in the presence of the target molecule, wherein the tandem antibody complex comprises the target molecule, the primary antibody, the second antibody, and the first solid phase, wherein the primary antibody binds to the target molecule, wherein the secondary antibody binds to the primary antibody, and wherein the first affinity label complexing agent of the first solid phase binds to the affinity label of the secondary antibody; (e) washing the binding composition comprising the tandem antibody complex with a washing solution; and, (f) eluting the composition comprising the target molecule isolated from the tandem antibody complex, to obtain the composition comprising the target molecule isolated from the sample.

In some aspects, provided herein is a method of isolating a composition comprising a target molecule from a sample, the method comprising: (a) crosslinking the sample; (b) lysing the crosslinked sample with a lysis solution; (c) heat denaturing the crosslinked lysate; (d) admixing the lysate with a first clearing agent and second clearing agent to form a pre-cleared sample lysate, wherein the first clearing agent comprises a second solid phase and an antibody binding agent that binds an antibody species, wherein the antibody species bound by the antibody binding agent is the same as the species of the primary antibody, and wherein the second clearing agent comprises a third solid phase and a second affinity label binding agent that binds the affinity label; (e) admixing the pre-cleared sample lysate with a primary antibody, a secondary antibody, and a first solid phase comprising a first affinity label binding agent, to form a binding composition, wherein a tandem antibody complex is formed in the binding composition in the presence of the target molecule, wherein the tandem antibody complex comprises the target molecule, the primary antibody, the second antibody, and the first solid phase, wherein the primary antibody binds to the target molecule, wherein the secondary antibody binds to the primary antibody, and wherein the first affinity label complexing agent of the first solid phase binds to the affinity label of the secondary antibody; (f) washing the binding composition comprising the tandem antibody complex with a washing solution; and, (g) eluting the composition comprising the target molecule isolated from the tandem antibody complex, to obtain the composition comprising the target molecule isolated from the sample.

In some embodiments, the sample contains, or is suspected of containing, a condensate. In some embodiments, the condensate is a cellular condensate. In some embodiments, the target molecule is, or can be, associated with a condensate, such as a cellular condensate.

In some aspects, provided herein is a method of isolating a composition comprising a target molecule from a sample, the method comprising: (a) contacting the sample with a compound (b) crosslinking the sample; (c) lysing the crosslinked sample with a lysis solution; (d) heat denaturing the crosslinked lysate; (e) admixing the lysate with a first clearing agent and second clearing agent to form a pre-cleared sample lysate, wherein the first clearing agent comprises a second solid phase and an antibody binding agent that binds an antibody species, wherein the antibody species bound by the antibody binding agent is the same as the species of the primary antibody, and wherein the second clearing agent comprises a third solid phase and a second affinity label binding agent that binds the affinity label; (f) admixing the pre-cleared sample lysate with a primary antibody, a secondary antibody, and a first solid phase comprising a first affinity label binding agent, to form a binding composition, wherein a tandem antibody complex is formed in the binding composition in the presence of the target molecule, wherein the tandem antibody complex comprises the target molecule, the primary antibody, the second antibody, and the first solid phase, wherein the primary antibody binds to the target molecule, wherein the secondary antibody binds to the primary antibody, and wherein the first affinity label complexing agent of the first solid phase binds to the affinity label of the secondary antibody; (g) washing the binding composition comprising the tandem antibody complex with a washing solution; and, (h) eluting the composition comprising the target molecule isolated from the tandem antibody complex, to obtain the composition comprising the target molecule isolated from the sample.

In some embodiments, the sample contains, or is suspected of containing, a condensate. In some embodiments, the condensate is a cellular condensate. In some embodiments, the target molecule is, or can be, associated with a condensate, such as a cellular condensate. In some embodiments, the compound is a small molecule (e.g., a drug). In some embodiments, the compound effects the condensate. In some embodiments, the method further comprises evaluating the differences in target molecule isolation in the presence of the compound compared to a reference sample not contacted with the compound.

In some aspects, provided herein is a method of isolating a composition comprising a target molecule from a sample, the method comprising: (a) contacting the sample with a compound (b) crosslinking the sample; (c) lysing the crosslinked sample with a lysis solution; (d) heat denaturing the crosslinked lysate for at least about 3 minutes and at a temperature of at least about 70° C.; (e) admixing the lysate with Pierce™ Protein A/G magnetic beads (e.g., a first clearing agent) and Pierce™ Streptavidin magnetic beads (e.g., a second clearing agent) to form a pre-cleared sample lysate, wherein the Pierce™ Protein A/G magnetic beads comprise a second solid phase (e.g., magnetic beads) and an antibody binding agent (e.g., Protein A/G) that binds an antibody species, wherein the antibody species bound by the antibody binding agent is the same as the species of the primary antibody, and wherein the second clearing agent comprises a third solid phase (e.g., magnetic beads) and a second affinity label binding agent (e.g., Streptavidin) that binds the affinity label; (f) admixing the pre-cleared sample lysate with a primary antibody, a biotinylated secondary antibody, and a first solid phase (e.g., magnetic beads) comprising a first affinity label binding agent (e.g., biotin), to form a binding composition, wherein a tandem antibody complex is formed in the binding composition in the presence of the target molecule, wherein the tandem antibody complex comprises the target molecule, the primary antibody, the second antibody, and the first solid phase, wherein the primary antibody binds to the target molecule, wherein the secondary antibody binds to the primary antibody, and wherein the first affinity label complexing agent of the first solid phase binds to the affinity label of the secondary antibody; (g) washing the binding composition comprising the tandem antibody complex with a washing solution; and, (h) eluting the composition comprising the target molecule isolated from the tandem antibody complex using an elution solution and heating, to obtain the composition comprising the target molecule isolated from the sample.

In certain aspects, the methods provided herein comprise an isolating step consisting essentially of isolating a composition comprising a target molecule via a tandem antibody complex of a target, a primary antibody, a second antibody, and a first solid phase. In some embodiments, provided is a method of isolating a composition comprising a target molecule from a sample, the method comprising: (a) admixing the sample with a primary antibody specifically recognizing the target molecule, a secondary antibody comprising an affinity label and specifically recognizing the primary antibody, and a first solid phase comprising a first affinity label complexing agent to form a binding composition, wherein a tandem antibody complex is formed in the binding composition in the presence of the target molecule, wherein the tandem antibody complex comprises the target, the primary antibody, the second antibody, and the first solid phase, wherein the primary antibody binds to the target molecule, wherein the secondary antibody binds to the primary antibody, and wherein the first affinity label complexing agent of the first solid phase forms a complex with the affinity label of the secondary antibody; and (b) an isolating step consisting essentially of isolating the composition comprising the target molecule via the tandem antibody complex of the target, the primary antibody, the second antibody, and the first solid phase.

Certain features and steps of the methods disclosed herein are described with additional detail in the sections below. The modular discussion of certain features and steps is not intended to limit the scope of the methods described herein, and using the teachings provided herein one can readily combine various modularly described features and steps to arrive at the full scope of the methods provided herein.

### A. Samples and Preparation Steps

### (i) Samples

The methods disclosed herein are useful for isolating a composition comprising a target molecule from a diverse array of samples. For example, in some embodiments, the methods described herein contain, or are suspected of containing, a polypeptide and/or nucleic acid target molecule. In some embodiments, the samples contains, or is suspected of containing, a condensate. In some embodiments, the condensate is a cellular condensate. In some embodiments, the target molecule is, or can be, associated with a condensate, such as a cellular condensate.

A sample disclosed herein can be, or can be derived from, any biological sample or in vitro system. Methods and compositions disclosed herein may be used for processing and/or analyzing a sample, such as a biological sample, which may be obtained from a subject using any of a variety of techniques including, but not limited to, biopsy, surgery, and laser capture microscopy (LCM), and generally includes cells and/or other biological material from the subject. In some embodiments, the sample is from an individual. In some embodiments, the individual is a mammal, such as a human, bovine, horse, feline, canine, rodent, or primate. In some embodiments, the method comprises obtaining the sample from an individual. In some embodiments, the methods and compositions disclosed herein may be used for processing and/or analyzing a sample, such as an in vitro system sample, which may be obtained from a cell system such as cultured cells.

In some embodiments, a biological sample can be obtained from (or comprise) a prokaryote, or a portion thereof, such as a bacterium, an archaea, a virus, or a viroid. In some embodiments, biological sample can be obtained from (or comprise) non-mammalian organisms, or a portion thereof, (e.g., a plant, an insect, an arachnid, a nematode, a fungus, or an amphibian). In some embodiments, a biological sample can be obtained from (or comprise) a eukaryote, or a portion thereof, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules and/or organelles and/or cellular structures, for example, cellular macromolecules and organelles (e.g., mitochondria and nuclei). In some embodiments, the biological sample is a nucleic acid sample and/or protein sample. In some embodiments, the biological sample is a carbohydrate sample or a lipid sample. In some embodiments, the biological sample is obtained as a tissue sample, such as a tissue section, biopsy, a core biopsy, needle aspirate, or fine needle aspirate. In some embodiments, the sample comprises a bodily fluid, such as a sample comprising a blood sample, serum sample, convalescent plasma sample, oropharyngeal sample, including that obtained from an oropharyngeal swab, nasopharyngeal sample, including that obtained from a nasopharyngeal swab, buccal sample, bronchoalveolar lavage sample, including that obtained from an endotracheal aspirator, a urine sample, a sweat sample, a sputum sample, a salivary sample, a tear sample, a bodily excretion sample, or cerebrospinal fluid sample. In some embodiments, the sample comprise a solid, such as a sample comprising a fecal sample. In some embodiments, the sample is a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions.

In some embodiments, the sample is a cell-free biological sample. In some embodiments, the cell-free biological samples can include extracellular polynucleotides, e.g., a cfDNA sample. Extracellular polynucleotides can be isolated from a bodily sample, e.g., blood, plasma, serum, urine, saliva, mucosal excretions, sputum, stool, and tears.

In some embodiments, biological samples are derived from a homogeneous culture or population of the subjects or organisms mentioned herein or alternatively from a collection of several different organisms, for example, in a community or ecosystem.

In some embodiments, biological samples may include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. In some embodiments, biological samples include fetal cells and immune cells.

In some embodiments, the sample comprises at least about 1×10⁷ cells/mL, such as at least about any of 1.5×10⁷ cells/mL, 2.0×10⁷ cells/mL, 2.1×10⁷ cells/mL, 2.2×10⁷ cells/mL, 2.3×10⁷ cells/mL, 2.4×10⁷ cells/mL, 2.5×10⁷ cells/mL, 2.6×10⁷ cells/mL, 2.7×10⁷ cells/mL, 2.8×10⁷ cells/mL, 2.9×10⁷ cells/mL, 3.0×10⁷ cells/mL, 3.5×10⁷ cells/mL, or 4.0×10⁷ cells/mL. In some embodiments, the sample comprises at least about 1×10⁷ cells, such as at least about any of 1.5×10⁷ cells, 2.0×10⁷ cells, 2.1×10⁷ cells, 2.2×10⁷ cells, 2.3×10⁷ cells, 2.4×10⁷ cells, 2.5×10⁷ cells, 2.6×10⁷ cells, 2.7×10⁷ cells, 2.8×10⁷ cells, 2.9×10⁷ cells, 3.0×10⁷ cells, 3.5×10⁷ cells, or 4.0×10⁷ cells.

In some embodiments, the sample comprises an environment sample, such as a sample comprising remains of in individual, such as a human or an animal, a food, a microorganism, a plant or its components, soil, sediment, rock, reef, sludge, decomposing biological matter, archaeological remains, oil, water, or air or particulates therein.

In some embodiments, the sample comprises a nucleic acid and/or a polypeptide, such as a peptide or protein. In some embodiments, nucleic acid comprises a RNA or DNA, or any combination thereof. In some embodiments the nucleic acid is a RNA or DNA. In some embodiments, the RNA is mRNA, tRNA, rRNA, or iRNA. In some embodiments, the RNA is a double-stranded viral RNA. In some embodiments, the RNA is a single-stranded viral RNA. In some embodiments, the RNA is negative-sense RNA, positive-sense RNA, or ambisense RNA.

(ii) Sample processing

In some embodiments, the sample is processed prior to subjecting to a purification method described herein. For example, in some embodiments, the sample is subjected to any one or more of a flocculation technique, a protein precipitation technique, a pathogen inactivation technique.

In some embodiments, the sample is crosslinked prior to purification analysis. In some embodiments, the sample is crosslinked prior to lysis with a lysis solution. Crosslinking may allow for the capture of transient interactions between target analytes, and/or the identification of low-abundance interactors. In some embodiments, the crosslinking is a reversible crosslinking. In some embodiments, the crosslinking comprises applying a crosslinker (e.g., paraformaldehyde, DSP, or any crosslinker known in the art) to the lysate. In some embodiments, the crosslinker is a thiol-cleavable crosslinker.

In some embodiments, the crosslinker is not affected by heat (e.g., the application of heat to the sample does not reverse the crosslinking). For example, the application of heat to the sample does not disrupt the crosslinking between protein-protein interactors in the sample. In some embodiments, the application of heat to the sample disrupts intra-molecular interactions but leaves crosslinked proteins in-tact. In some embodiments, the method comprises de-crosslinking the reversibly crosslinked biological sample. In some embodiments, the de-crosslinking does not need to be complete. In some embodiments, only a portion of crosslinked molecules in the reversibly crosslinked biological sample are de-crosslinked.

In some embodiments, the sample is treated with a compound prior to crosslinking. In some embodiments, the compound is a pharmaceutical agent, such as but not limited to, a small molecule. For example, the compound may have an effect on a target molecule. In some embodiments, the sample is treated with a compound and crosslinked, e.g., to investigate the impact of the compound on a target molecule, e.g., to investigate the impact of the compound on a condensate.

In some embodiments, the sample is lysed prior to subjecting to an isolation method described herein. In some embodiments, the lysis comprises adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes. In some embodiments, other lysis agents can additionally or alternatively be added to the biological sample to facilitate lysis prior to co-IP. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the lysis comprises a physical lysis technique. In some embodiments, the physical lysis comprises a non-chemical lysis technique. In some embodiments, the physical lysis comprises electroporation, mechanical permeabilization methods (e.g., bead beating using a homogenizer and grinding balls to mechanically disrupt sample tissue structures), acoustic permeabilization (e.g., sonication), or thermal lysis techniques such as heating.

In some embodiments, the lysis agents may further comprise detergents (e.g., saponin, Triton X-100™, Tween-20™, or NP-40). In some embodiments, additional reagents are added to the lysis solution, or the sample, to perform various functions prior to co-IP analysis of the sample. In some embodiments, DNase and RNase inactivating agents or inhibitors such as proteinase K, phosphatase inhibitors, protease inhibitors, and/or chelating agents such as EDTA, can be added to the sample.

In some embodiments, the method described herein further comprises denaturing the sample (e.g., the crosslinked lysate) prior to analysis of the sample. In some embodiments, the sample is denatured following crosslinking. In some embodiments, the samples is denatured following lysis. In some embodiments, the sample is denatured following crosslinking and lysis. For example, the crosslinked lysate may be denatured. In some embodiments, the denaturing is a partial denaturing, such that portions of the secondary structure of the target and/or the crosslinking remains in-tact. In some embodiments, the denaturing comprises chemical denaturing or heat denaturing. In some embodiments, the denaturation disrupts hydrogen bonds and non-polar hydrophobic interactions, but preserves the crosslinked interactions (e.g., disulfide bonds) held together by the crosslinker. In some embodiments, the denaturing disrupts the quaternary, tertiary, and secondary structures of proteins within the same. In some embodiments, the heat denaturing comprises heating the lysate at a temperature greater than about 70° C., such as greater than about any of 75° C., 80° C., 85° C., 90° C., 95° C., and 100° C. In some embodiments, the heat denaturing comprises heating the lysate at a temperature of about 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C. In some embodiments, the sample is subjected to heat for at least about 3 minutes, such as at least about any of 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, and 20 minutes. In some embodiments, the sample is subjected to heat for about 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, or 20 minutes. In some embodiments, the lysate is heated for at least about 3 minutes and at least about 90° C. In some embodiments, the lysate is heated for about 5 minutes at about 95° C. In some embodiments, the denaturing increases access of the epitope binding site in crosslinked lysate, enabling IP on previously unusable antibodies. In some embodiments, the heat denaturing is followed by immediate transition to ice to prevent renaturation/refolding.

(iii) Target Molecules and Compositions Thereof

The methods disclosed herein can be used to isolate a wide variety of different target molecules and compositions comprising a target molecule. In some embodiments, the composition comprising the target molecule comprises another molecule, such as one that co-isolated with the target molecule. In some aspects, the target molecule is any biological substance, structure, moiety, or component to be analyzed. In some aspects, the target molecules disclosed herein may similarly include any target molecule of interest. For example, in some embodiments, the methods described herein contain, or are suspected of containing, a polypeptide and/or nucleic acid target molecule. In some embodiments, the samples contains, or is suspected of containing, a condensate. In some embodiments, the condensate is a cellular condensate. In some embodiments, the target molecule is, or can be, associated with a condensate, such as a cellular condensate.

In some embodiments, the target molecule is derived from a specific type of cell and/or a specific sub-cellular region. For example, the target molecule can be derived from cytosol, from cell nuclei, from mitochondria, from microsomes, and more generally, from any other compartment, organelle, or portion of a cell. Permeabilizing agents that specifically target certain cell compartments and organelles can be used to selectively release target molecules from cells for analysis, and/or allow access of one or more reagents (e.g., clearing agents and/or primary and secondary antibodies) to the target molecules in the cell or cell compartment or organelle.

The target molecule may include any biomolecule or chemical compound, including a macromolecule such as a protein or peptide, a lipid or a nucleic acid molecule, or a small molecule, including organic or inorganic molecules. In some embodiments, the target molecule may be comprised in an analyte a cell or a microorganism, including a virus, or a fragment or product thereof.

In some embodiments, the target molecule is a nucleic acid molecule, such as DNA (e.g., genomic DNA, mitochondrial DNA, plastid DNA, and viral DNA) and/or RNA (e.g., mRNA, microRNA, rRNA, snRNA, and viral RNA). In some embodiments, the nucleic acid molecule is a synthetic and/or modified nucleic acid molecule, (e.g., including nucleic acid domains comprising or consisting of synthetic or modified nucleotides such as LNA, PNA, and morpholino). In some embodiments, the target molecule comprises proteinaceous molecules, such as peptides, polypeptides, proteins or prions or any molecule which includes a protein or polypeptide component, or fragments thereof. In some embodiments, the target molecule is a lipid or carbohydrate molecule, or any molecule which comprise a lipid or carbohydrate component.

In some embodiments, the target molecule is a single molecule, such as a single polypeptide. In some embodiments, the target molecule is associated with other components (e.g., other cellular components interacting with the target molecule such as co-binders), and may be co-isolated with said co-binders by the methods described herein. Generally speaking, co-binders are molecules that interact with the target molecule in a biological setting, such as in vitro and/or in vivo, and such interactions are not purely a product of the isolation process, e.g., an artifact produced during an isolation method or sample processing technique. Rather, in some embodiments, the method provided herein preserve the biological interaction of a co-binder and a target molecule, and thus via isolation of the target molecule using the methods provided herein the co-binder can also be isolated. In some embodiments, the co-binder interacts with the target molecule in a sample, such as a biological sample. In some embodiments, the co-binders interact with the target molecule prior to a sample processing step.

In some embodiments, the antibody (e.g., a primary antibody) is based on the target molecule of interest. In some embodiments, the binding agent, such as a primary antibody, used in the isolation methods taught herein is based on a characteristic of a target molecule and interactions associated with the target molecule. In some embodiments, the target molecule is, or is part of, a complex such as complex that contains two or more molecular subunits, e.g., a protein-DNA complexes, which may be covalently bound to or non-covalently associated with one another, or a polypeptide-polypeptide complex. Thus, such a complex target molecule may be a protein complex or protein interaction. Such a complex or interaction may thus be a homo- or hetero-multimer. In some embodiments, multi-specific binders, such as bi-specific antibodies, can be used in the methods described herein, such as to bind to complexes. Aggregates of molecules, e.g. proteins, may also be target molecules, for example aggregates of the same protein or different proteins. The target molecule may also be a complex between proteins or peptides and nucleic acid molecules such as DNA or RNA, e.g. interactions between proteins and nucleic acids, e.g. regulatory factors, such as transcription factors, and DNA or RNA. In some embodiments, the target molecule is a complex, a polypeptide, a polypeptide complex, or a mixed complex.

In some embodiments, the target molecule is associated with a condensate, or a component thereof, such as a polypeptide component of a biomolecular condensate. In some embodiments, the target is a polypeptide that is, or can be, associated with a condensate. In some embodiments, the target is a nucleic acid that is, or can be, associated with a condensate. In some embodiments, the condensate is a nuclear, cytoplasmic, plasma membrane associated, or secreted extracellular condensate. In some embodiments, methods described herein are capable of isolating molecules involved in transient interactions of a target molecule within the condensate context. For example, in some embodiments, the method described herein is useful for isolating a target molecule associated with additional molecules, i.e., the target molecule is co-isolated with additional associated molecules.

### B. Crosslinking

As described herein, in certain aspects, the method comprise use of a crosslinker in one or more crosslinking steps. In some embodiments, the crosslinker is useful for capturing proteins in a biologically relevant state, such as prior to changes caused by sample handling and/or preparation. For example, condensates are dynamic and changes in composition can occur during sample handling and/or preparation leading to non-biologically relevant artifact generation. Crosslinking may allow for the capture of weak and/or transient interactions between target analytes, and/or the identification of low-abundance interactors.

In some embodiments, the sample is crosslinked prior to a purification analysis described herein. In some embodiments, the sample is crosslinked prior to lysis with a lysis solution.

In some embodiments, the crosslinker is a reversible crosslinker. In some embodiments, the crosslinking comprises applying a crosslinker to a sample, such as a lysate. In some embodiments, the crosslinker is a thiol-cleavable crosslinker (e.g., can be cleaved using conventional disulfide separation techniques). In some embodiments, the crosslinker is dithiobis(succinimidyl propionate) (DSP). In some embodiments, the method comprises a step that cleaves the crosslinker, such as an elution step comprising heat and/or a reducing agent (e.g., TCEP).

In some embodiments, the crosslinker is not affected by heat (e.g., the application of heat to the sample does not reverse the crosslinking). For example, the application of heat to the sample does not disrupt the crosslinking between protein-protein interactors in the sample. In some embodiments, the application of heat to the sample disrupts intra-molecular interactions but leaves crosslinked proteins intact. In some embodiments, the method comprises de-crosslinking the reversibly crosslinked biological sample. In some embodiments, the de-crosslinking does not need to be complete. In some embodiments, only a portion of crosslinked molecules in the reversibly crosslinked biological sample are de-crosslinked.

In some embodiments, the sample is treated with a compound prior to crosslinking. In some embodiments, the compound is a pharmaceutical agent, such as but not limited to, a small molecule. For example, the compound may have an effect on a target molecule. In some embodiments, the sample is treated with a compound and crosslinked, e.g., to investigate the impact of the compound on a target molecule, e.g., to investigate the impact of the compound on a condensate.

In some embodiments, the concentration of a crosslinker added to a sample, such as a sample comprising cells, is about 1 mM or less, such as about any of 0.9 mM or less, 0.85 mM or less, 0.8 mM or less, 0.75 mM or less, 0.7 mM or less, 0.65 mM or less, 0.6 mM or less, 0.55 mM or less, 0.5 mM or less, 0.45 mM or less, 0.4 mM or less, 0.35 mM or less, 0.3 mM or less, 0.25 mM or less, 0.2 mM or less, 0.15 mM or less, or 0.1 mM or less. In some embodiments, the concentration of a crosslinker added to a sample, such as a sample comprising cells, is about 0.095 mM or less, such as about any of 0.090 mM or less, 0.085 mM or less, 0.080 mM or less, 0.075 mM or less, 0.070 mM or less, 0.065 mM or less, 0.060 mM or less, 0.055 mM or less, 0.050 mM or less, 0.045 mM or less, 0.040 mM or less, 0.035 mM or less, 0.030 mM or less, 0.025 mM or less, 0.020 mM or less, 0.015 mM or less, or 0.010 mM or less. In some embodiments, provided is a method for determining a crosslinker amount for use in the methods taught herein, the method comprising assessing proteins captured using a plurality of crosslinker amounts and determining the amount of cross linker based on results therefrom, such as bait protein amount (e.g., as determined from bait protein mass spectrometry intensities), and/or quantity and/or number of known condensate proteins identified/captured (such as identified from mass spectrometry).

### C. Pre-Clearing

In some aspects, the methods described herein comprise a pre-clearing step. In some embodiments, the pre-clearing step is based on the primary and/or secondary antibodies used to form a tandem antibody complex. The sample used for the provided methods may therefore be a pre-cleared sample that has been obtained via a pre-clearing step.

In some embodiments, the pre-clearing step comprises admixing the sample with one or more clearing agents. In some embodiments, the pre-clearing step is a two-part pre-clearing step, wherein the two-part pre-clearing step comprises admixing the sample with a first clearing agent and a second clearing agent. In some embodiments, the sample is admixed with the first clearing agent and the second clearing agent simultaneously or concurrently. In some embodiments, the sample is admixed with the first clearing agent prior to the second clearing agent. In some embodiments, the sample is admixed with the second clearing agent prior to the first clearing agent.

In some embodiments, the first clearing agent comprises a solid phase (e.g., a second solid phase) and an antibody binding agent that binds to a population of antibodies. In some embodiments, the population of antibodies comprises antibodies identical to that of a primary antibody, wherein the primary antibody binds to a target molecule in the sample. In some embodiments, the primary antibody belongs to the population of antibodies bound by the antibody binding agent of the first clearing agent. In some embodiments, the antibody binding agent of the first clearing agent forms a complex with the population of antibodies in the same prior to contacting the sample with the primary antibody, to allow for increased reaction specificity between the primary antibody and a target molecule during IP isolation of a target molecule.

In some embodiments, the population of antibodies bound by the first clearing agent is characterized by the organism of origin. For example, the first clearing agent may bind selectively to a population of antibodies derived from a specific organism of origin. In some embodiments, the population of antibodies bound by the first clearing agent originate from a human, rabbit, mouse, or pig. In some embodiments, the population of antibodies bound by the first clearing agent is characterized by the antibody isotype. For example, the first clearing agent may bind selectively to a population of antibodies derived from a specific antibody isotype. In some embodiments, the antibody isotope of the population of antibodies bound by the first clearing agent is selected from the group consisting of IgG, IgM, IgD, IgE, and IgA. In some embodiments, the antibody isotope of the population of antibodies bound by the first clearing agent is IgG. In some embodiments, the IgG is selected from the group consisting of IgG1, IgG2, IgG3, and IgG4. In some embodiments, the antibody isotope of the population of antibodies bound by the first clearing agent is IgA. In some embodiments, the IgA is selected from the group consisting of IgA1 and IgA2. In some embodiments, the antibody binding agent of the first clearing agent comprises protein G, protein A, protein A/G, protein L, or jacalin. In some embodiments, the antibody binding agent of the first clearing agent comprises protein G. In some embodiments, the protein G antibody binding agent of the first clearing agent binds IgG, such as any of the IgGs described above. In some embodiments, the antibody binding agent of the first clearing agent comprises protein A. In some embodiments, the protein A antibody binding agent of the first clearing agent binds IgA, such as any of the IgAs described above. In some embodiments, the protein A/G antibody binding agent of the first clearing agent binds IgG and IgA, such as any of the IgGs and IgAs described above.

In some embodiments, the second clearing agent comprises a solid phase (e.g., a third solid phase) and an affinity label complexing agent (e.g., a second affinity label complexing agent). In some embodiments, the second affinity label complexing agent forms a complex with an affinity label. In some embodiments, the second affinity label complexing agent forms a complex with an affinity label in a free state. In some embodiments, the affinity label that forms a complex with the second affinity label complexing agent is identical to the affinity label of a secondary antibody described herein. In some embodiments, the second affinity label complexing agent of the second clearing agent forms a complex with affinity label in the sample prior to contacting the sample with the secondary antibody comprising the affinity label, to allow for increased reaction specificity between the secondary antibody and a solid phase during IP isolation of a target molecule.

In some embodiments, the second affinity label complexing agent of the second clearing agent comprises streptavidin, an anti-biotin antibody, or NeutrAvidin. In some embodiments, the second affinity label complexing agent comprises streptavidin.

In some embodiments, the first clearing agent comprises a solid phase (e.g., a second solid phase) that is the same as the solid phase of the second clearing agent (e.g., a third solid phase). In some embodiments, the second solid phase and the third solid phase are different. In some embodiments, the second solid phase and the third solid phase comprise a bead. In some embodiments, the bead is a magnetic bead. In some embodiments, the bead is a non-magnetic bead. In some embodiments, the non-magnetic beads are configured in a chromatography column.

In some embodiments, the first clearing agent and the second clearing agent (e.g., the first clearing agent complexed with the population of antibodies and the second clearing agent complexed with the affinity label) are separated from the sample comprising the target molecule to obtain the pre-cleared sample. In some embodiments, the separating of the first clearing agent and the second clearing agent from the sample occurs after a period of incubation. In some embodiments, the period of incubation is sufficient to allow the first clearing agent and/or the second clearing agent to form a complex with the population of antibodies and/or the affinity label, respectively. In some embodiments, the sample is incubated with the first clearing agent and the second clearing agent for about 15 minutes or more, such as about any of 20 minutes or more, 25 minutes or more, 30 minutes or more, 35 minutes or more, 40 minutes or more, 45 minutes or more, 50 minutes or more, 55 minutes or more, or 60 minutes or more.

In some embodiments, the separating of the first clearing agent and/or the second clearing agent comprises pelleting the first clearing agent and/or the second clearing agent. In some embodiments, the pelleting comprises magnetic separation, such as wherein the solid phase of the first and/or second clearing agent comprises magnetic beads. In some embodiments, the pelleting comprises centrifugation or use of a packed particulate column. In some embodiments, the separating of the first clearing agent and/or the second clearing agent comprises repeating the incubation of the first clearing agent and/or the second clearing agent with the sample and separating with a second round of pelleting (e.g., magnetic pelleting or centrifugation) to obtain the pre-cleared sample. In some embodiments, the separating of the first clearing agent and/or the second clearing agent comprises only one round of incubation and pelleting separation to obtain the pre-cleared sample.

### D. Target Isolation and Reagents Useful Therefor

In some aspects, the method provided herein comprises a target molecule isolation step, such as a pull down step to obtain a composition comprising target molecule from a sample. The target isolation may be mediated by a primary antibody and a secondary antibody reagent, or a component thereof, wherein the primary antibody and secondary antibody form a tandem antibody complex comprising the target molecule and a solid phase (e.g., a first solid phase). In some embodiments, the sample is admixed with a primary antibody, a secondary antibody, and a solid phase (e.g., a first solid phase) to form a binding composition. In some embodiments, the sample is admixed with the primary antibody, the secondary antibody, and the first solid phase simultaneously to form a binding composition. In some embodiments, the sample is a pre-cleared sample.

The primary antibody specifically recognizes and binds to a target molecule. In some embodiments, the primary antibody is any construct that is capable of binding to a target molecule. In some embodiments, the primary antibody is selected based on the specific class of target molecule (e.g., nucleic acid target molecule or protein target molecule). In some embodiments, the primary antibody is capable of hybridizing to the target molecule. In some embodiments, the primary antibody is a fusion construct. For example, the primary antibody may be a nucleic acid-polypeptide fusion. In some embodiments, the nucleic acid-polypeptide fusion hybridizes with the target molecule via the nucleic acid portion. In some embodiments, the primary antibody binds to a non-conformational epitope of a target molecule, e.g., an amino acid sequence. In some embodiments, the primary antibody binds to a conformational epitope of a target molecule, e.g., a structure of the target molecule in a folded state.

In some embodiments, the primary antibody is characterized by the antibody isotype. In some embodiments, the antibody isotope of the primary antibody is selected from the group consisting of IgG, IgM, IgD, IgE, and IgA. In some embodiments, the antibody isotope of the primary antibody is IgG. In some embodiments, the IgG is selected from the group consisting of IgG1, IgG2, IgG3, and IgG4. In some embodiments, the antibody isotope of the primary antibody is IgA. In some embodiments, the IgA is selected from the group consisting of IgA1 and IgA2.

In some embodiments, the primary antibody binds with a secondary antibody. In some embodiments, the secondary antibody specifically recognizes the primary antibody. In some embodiments, the secondary antibody binds to a polypeptide region of the primary antibody. In some embodiments, the secondary antibody binds to the Fc region of the primary antibody.

In some embodiments, the secondary antibody further comprises an affinity label. In some embodiments, the affinity label comprises a ligand. In some embodiments, the ligand is biotin. In some embodiments, the secondary antibody is capable of binding with the primary antibody (e.g., via the Fc region) and a first solid phase. In some embodiments, the first solid phase comprises a first affinity label complexing agent. In some embodiments, the first affinity label complexing agent of the first solid phase is the same second affinity label complexing agent of the second clearing agent. In some embodiments, the first affinity label complexing agent of the first solid phase is different than the second affinity label complexing agent of the second clearing agent.

In some embodiments, the first affinity label complexing agent of the first solid phase comprises a receptor of the ligand of the secondary antibody. In some embodiments, the affinity label of the secondary antibody comprises biotin, and the first affinity label complexing agent of the first solid phase comprises streptavidin. In some embodiments, the affinity label of the secondary antibody comprises biotin, and the first affinity label complexing agent of the first solid phase comprises anti-biotin.

In some embodiments, the affinity label of the secondary antibody is a portion of a reactive group. In some embodiments, the first affinity label complexing agent of the first solid phase comprises a portion of a reactive group. In some embodiments, the reactive groups of the affinity label and the first affinity label complexing agent are complementary click functional groups (e.g., capable of reacting with each other). In some embodiments, the affinity label of the secondary antibody forms a complex with the first affinity label complexing agent of the first solid phase via click chemistry. In some embodiments, the affinity label click functional group and the affinity label complexing agent click functional group are an alkynyl and a azido, an azido and a cyclooctyny, a tetrazine and a dienophile, a thiol and a alkynyl, or any other suitable functional group pairing known in the art.

In some embodiments, the primary antibody, the secondary antibody, and the first solid phase form a binding composition. In some embodiments, the binding composition forms a tandem antibody complex in the presence of a target molecule. In some embodiments, the tandem antibody complex comprises the target molecule, the primary antibody, the secondary antibody, and the first solid phase. In some embodiments, the primary antibody binds to the target molecule, the secondary antibody binds to the primary antibody (e.g., via the Fc region of the primary antibody), and the first affinity label complexing agent of the first solid phase forms a complex with the affinity label of the secondary antibody.

In some embodiments, the primary antibody is added to a pre-cleared sample. In some embodiments, the primary antibody is added to a sample that has not been pre-cleared. In some embodiments, the sample is incubated with the primary antibody for a sufficient time such that the primary antibody binds to the target molecule. In some embodiments, the sample is incubated with the primary antibody for a period comprising up to about 24 hours, such as up to any of about 23 hours, 22 hours, 21 hours, 20 hours, 19 hours, 18 hours, 17 hours, 16 hours, 15 hours, 14 hours, 13 hours, 12 hours, 11 hours, 10 hours, 9 hours, 8 hours, 7 hours, 6 hours, 5 hours, 4 hours, 3 hours, 2 hours, or 1 hour. In some embodiments, the sample is incubated with the primary antibody overnight. In some embodiments, the incubation occurs at a temperature of about 4° C., such as any of about 2° C., 3° C., 4° C., 5° C., or 6° C. In some embodiments, the incubation occurs at 4° C. In some embodiments, the primary antibody binds to the target molecule during the incubation with the sample.

In some embodiments, the sample is incubated with a secondary antibody comprising an affinity label, following incubation with the primary antibody. In some embodiments, the secondary antibody specifically binds to the primary antibody (e.g., via the Fc region of the primary antibody). In some embodiments, the sample is incubated with the secondary antibody for a sufficient time such that the secondary antibody binds to the primary antibody. In some embodiments, the sample is incubated with the secondary antibody for a period comprising up to about 5 hours, such as up to any of about 4 hours, 3 hours, 2 hours, 1 hour, 30 minutes, or 15 minutes. In some embodiments, the sample is incubated with the secondary antibody for 1 hour. In some embodiments, the incubation occurs at a temperature of about 4° C., such as any of about 2° C., 3° C., 4° C., 5° C., or 6° C. In some embodiments, the incubation occurs at 4° C. In some embodiments, the secondary antibody binds to the primary antibody during the incubation with the sample. In some embodiments, the primary antibody binds to the target molecule before the secondary antibody binds to the primary antibody. In some embodiments, the secondary antibody binds to the primary antibody before the primary antibody binds to the target molecule. In some embodiments, the secondary antibody binds to the primary antibody concurrently with the primary antibody binding to the target molecule.

In some embodiments, the sample is incubated with a first solid phase comprising a first affinity label complexing agent, following incubation with a primary antibody and a secondary antibody, to form a binding composition. In some embodiments, the affinity label complexing agent of the first solid phase forms a complex with the affinity label of the secondary antibody. In some embodiments, the sample is incubated with the first solid phase for a sufficient time such that the first solid phase forms a complex with the secondary antibody. In some embodiments, the sample is incubated with the first solid phase for a period comprising up to about 5 hours, such as up to any of about 4 hours, 3 hours, 2 hours, 1 hour, 30 minutes, or 15 minutes. In some embodiments, the sample is incubated with the first solid phase for a period comprising any of about 15 minutes, 30 minutes, 1 hour, 2 hours, 3 hours, 4 hours, or 5 hours. In some embodiments, the sample is incubated with the first solid phase for about 1 hour. In some embodiments, the incubation occurs at a temperature of about 4° C., such as any of about 2° C., 3° C., 4° C., 5° C., or 6° C. In some embodiments, the incubation occurs at a temperature of any of about 2° C., 3° C., 4° C., 5° C., or 6° C. In some embodiments, the incubation occurs at about 4° C. In some embodiments, the first solid phase forms a complex with the secondary antibody (e.g., the first affinity label complexing agent of the first solid phase forms a complex with the affinity label of the secondary antibody) during the incubation with the sample. In some embodiments, the secondary antibody binds to the primary antibody and/or the primary antibody binds to the target molecule before the first solid phase forms a complex with the secondary antibody. In some embodiments, the first solid phase forms a complex with the secondary antibody before the secondary antibody binds to the primary antibody and/or the primary antibody binds to the target molecule. In some embodiments, the first solid phase, the secondary antibody, the primary antibody, and the target molecule form a tandem antibody complex in the binding composition.

### E. Washing and Elution

In some aspects, the method described herein further comprises a washing step to isolate a tandem antibody complex comprising a primary antibody, a secondary antibody, a first solid phase, and a target molecule from a binding composition. In some embodiments, the method further comprises washing the binding composition comprising the tandem antibody complex. In some embodiments, the method further comprises eluting a target molecule, and components associated therewith, from the tandem antibody complex comprising the target molecule, to obtain the target molecule.

In some embodiments, the tandem antibody complex is isolated from the binding composition by separating the tandem antibody complex from other components of the binding composition (e.g., unbound primary antibodies, unbound secondary antibodies, unbound first solid phase, and/or non-target molecules in the sample). In some embodiments, the separating comprises a washing step. In some embodiments, the washing step comprises pelleting the binding composition, removing the resulting supernatant, adding the washing solution, pelleting a resulting solution, and removing a resulting solution. In some embodiments, the washing solution comprises a buffer solution that is similar to (e.g., compatible with) that of the lysis solution used to lyse the sample prior to sample pre-clearing and/or IP.

In some embodiments, the pelleting comprises centrifugation of the binding composition. In some embodiments, the pelleting comprises a magnetic separation of the tandem antibody complex from the binding composition (e.g., wherein the first solid phase comprises magnetic beads). In some embodiments, the pelleting of the tandem antibody complex (e.g., the first solid phase comprising the tandem antibody complex) concentrates the compositions comprising the target molecule (e.g., the tandem antibody complex) in the pellet. In some embodiments, the supernatant is removed from the pelleted first solid phase comprising the tandem antibody complex. In some embodiments, the removing of the supernatant produces a solution of concentrated tandem antibody complex comprising the target molecule. In some embodiments, the washing step is repeated at least once. In some embodiments, the washing step is repeated twice, three times, four times, or five times. In some embodiments, the washing step is repeated sufficiently to concentrate the first solid phase comprising the tandem antibody complex in the pellet.

In some embodiments, the target molecule, and components associated therewith, is eluted from the first solid phase. In some embodiments, the target molecule is eluted using an elution solution. In some embodiments, the elution from the first solid phase does not disrupt the association between the primary antibody and the target molecule. In some embodiments, the elution from the first solid phase disrupts the association between the primary antibody and the target molecule. In some embodiments, the elution solution comprises a ligand that displaces (e.g., competes with binding) the tandem antibody complex from the first solid phase. In some embodiments, the ligand is the same as the affinity label of the secondary antibody. In some embodiments, the ligand is biotin. In some embodiments, the elution solution comprises at least about 2 nM of biotin, such as any of about 3 nM, 4 nM, 5 nM, or 10 nM of biotin. In some embodiments, the elution occurs at room temperature (e.g., about 23° C.). In some embodiments, the elution occurs at an elevated temperature (e.g., heated). For example, in some embodiments, the elution occurs at least at about 70° C., such as at least about any of 75° C., 80° C., 85° C., 90° C., 95° C., or 100° C., In some embodiments, the elution solution is heated prior to application to the sample.

In some embodiments, the target molecule, and components associated therewith, is eluted from the first solid phase by contacting the pelleted binding composition with a reducing elution solution. In some embodiments, the elution solution comprises a reducing agent. In some embodiments, the elution solution is a sample buffer for downstream analyses, such as Laemmli buffer with a reducing agent such as tris(2-carboxyethyl) phosphine) (TCEP). In some embodiments, the eluting comprises heating the pelleted binding composition after applying the elution solution. In some embodiments, the pelleted binding composition is heated at about 95° C. for about 5 min in the presence of an elution solution to obtain the eluted target molecule. In some embodiments, the eluting comprises only heating. In some embodiments, wherein the eluting comprises only heating, the heating is performed for a duration longer than about 5 minutes without the presence of an elution solution. In some embodiments, the eluting comprises only subjecting the pelleted binding composition to an elution solution. In some embodiments, the eluting comprises both heating and subjecting the pelleted binding composition to an elution solution.

Carryover of antibody from immunoprecipitation may affect detection of low abundance samples by mass spectrometry. This is because of the relative abundance of antibody versus proteins of interest. In some embodiments, the eluent is subjected to a protease digestion. In some embodiments the eluent is subject to protease digestion prior to subjecting the sample to mass spectrometry. In some embodiments, the bait-prey protein complex is eluted from a solid phase by on-bead protease digestion. In some embodiments, on-bead protease digestion comprises the use of trypsin (see, e.g., Mohammed et al., Nature Protocols, Vol. 11 No. 2, 2016, p. 316, which is hereby incorporated by reference in its entirety).

In some embodiments, the eluting further comprises centrifugation or magnetic separation to separate the target molecule from the first solid phase. In some embodiments, the primary antibody remains associated with the target molecule during the eluting step. In some embodiments, the interaction between the primary antibody and the target molecule is broken during the eluting step. In some embodiments, the target molecule, and components associated therewith, is obtained with about 20-95% recovery after eluting.

### F. Additional Methods and Method Steps

In certain aspects, the methods described herein are integrated with one or more upstream and/or downstream techniques. In some embodiments, the eluate obtained from an isolation method described herein is processed to identify and, if desired, quantify target molecules, and components associated therewith, in the eluate. In some embodiments, the method comprises evaluating the purity and/or recovery of the target molecule. In some embodiments, the purity and/or recovery of the target molecule is assessed by SDS-PAGE, immunoblot, mass spectrometry, or liquid-chromatography/mass spectrometry. In some embodiments, the method comprises assessing whether the sample contains the presence (or lacks the presence) of a certain target molecule.

### G. Exemplary Methods

Exemplary isolation methods provided herein are described below. Such description of specific isolation methods in this section is not to be construed as limiting the methods encompassed by the disclosure of the instant application.

In some aspects, provided is a method of isolating a composition comprising a target molecule from a sample, the method comprising: (a) admixing the sample with a primary antibody specifically recognizing the target molecule, a secondary antibody comprising an affinity label and specifically recognizing the primary antibody, and a first solid phase comprising a first affinity label complexing agent to form a binding composition, wherein a tandem antibody complex is formed in the binding composition in the presence of the target molecule, wherein the tandem antibody complex comprises the target, the primary antibody, the second antibody, and the first solid phase, wherein the primary antibody binds to the target molecule, wherein the secondary antibody binds to the primary antibody, and wherein the first affinity label complexing agent of the first solid phase forms a complex with the affinity label of the secondary antibody; and (b) isolating the composition comprising the target molecule, wherein isolating the composition comprising the target molecule does not comprise formation of a second tandem antibody complex comprising the primary antibody, the secondary antibody, and a solid phase. In some embodiments, the isolating step comprises a target molecule pull-down step, wherein the pull-down step comprises isolating the first solid phase, and components associated therewith, from other components in the sample or the pre-cleared sample, and wherein the method does not comprise a second target molecule pull-down step.

In some aspects, provided is a method of isolating a composition comprising a target molecule from a sample, the method comprising: (a) obtaining a pre-cleared sample by admixing the sample with first clearing agent and a second clearing agent prior to form the pre-cleared sample, wherein the first clearing agent comprises a second solid phase and an antibody binding agent that binds to a population of antibodies, and wherein the population of antibodies comprises the primary antibody specifically recognizing the target molecule, wherein the second clearing agent comprises a third solid phase and a second affinity label complexing agent that forms a complex with the affinity label in a free state; (b) admixing the sample with a primary antibody specifically recognizing the target molecule, a secondary antibody comprising an affinity label and specifically recognizing the primary antibody, and a first solid phase comprising a first affinity label complexing agent to form a binding composition, wherein a tandem antibody complex is formed in the binding composition in the presence of the target molecule, wherein the tandem antibody complex comprises the target, the primary antibody, the second antibody, and the first solid phase, wherein the primary antibody binds to the target molecule, wherein the secondary antibody binds to the primary antibody, and wherein the first affinity label complexing agent of the first solid phase forms a complex with the affinity label of the secondary antibody; and (c) isolating the composition comprising the target molecule, wherein isolating the composition comprising the target molecule does not comprise formation of a second tandem antibody complex comprising the primary antibody, the secondary antibody, and a solid phase. In some embodiments, the isolating step comprises a target molecule pull-down step, wherein the pull-down step comprises isolating the first solid phase, and components associated therewith, from other components in the sample or the pre-cleared sample, and wherein the method does not comprise a second target molecule pull-down step.

In some aspects, provided is a method of isolating a composition comprising a target molecule from a sample, the method comprising: (a) crosslinking the sample; (b) denaturing the crosslinked sample; (c) admixing the sample with a primary antibody specifically recognizing the target molecule, a secondary antibody comprising an affinity label and specifically recognizing the primary antibody, and a first solid phase comprising a first affinity label complexing agent to form a binding composition, wherein a tandem antibody complex is formed in the binding composition in the presence of the target molecule, wherein the tandem antibody complex comprises the target, the primary antibody, the second antibody, and the first solid phase, wherein the primary antibody binds to the target molecule, wherein the secondary antibody binds to the primary antibody, and wherein the first affinity label complexing agent of the first solid phase forms a complex with the affinity label of the secondary antibody; and (b) isolating the composition comprising the target molecule, wherein isolating the composition comprising the target molecule does not comprise formation of a second tandem antibody complex comprising the primary antibody, the secondary antibody, and a solid phase. In some embodiments, the isolating step comprises a target molecule pull-down step, wherein the pull-down step comprises isolating the first solid phase, and components associated therewith, from other components in the sample or the pre-cleared sample, and wherein the method does not comprise a second target molecule pull-down step.

In some aspects, provided is a method of isolating a composition comprising a target molecule from a sample, the method comprising: (a) crosslinking the sample; (b) denaturing the crosslinked sample; (c) obtaining a pre-cleared sample by admixing the sample with first clearing agent and a second clearing agent prior to form the pre-cleared sample, wherein the first clearing agent comprises a second solid phase and an antibody binding agent that binds to a population of antibodies, and wherein the population of antibodies comprises the primary antibody specifically recognizing the target molecule, wherein the second clearing agent comprises a third solid phase and a second affinity label complexing agent that forms a complex with the affinity label in a free state; (d) admixing the sample with a primary antibody specifically recognizing the target molecule, a secondary antibody comprising an affinity label and specifically recognizing the primary antibody, and a first solid phase comprising a first affinity label complexing agent to form a binding composition, wherein a tandem antibody complex is formed in the binding composition in the presence of the target molecule, wherein the tandem antibody complex comprises the target, the primary antibody, the second antibody, and the first solid phase, wherein the primary antibody binds to the target molecule, wherein the secondary antibody binds to the primary antibody, and wherein the first affinity label complexing agent of the first solid phase forms a complex with the affinity label of the secondary antibody; and (e) isolating the composition comprising the target molecule, wherein isolating the composition comprising the target molecule does not comprise formation of a second tandem antibody complex comprising the primary antibody, the secondary antibody, and a solid phase. In some embodiments, the isolating step comprises a target molecule pull-down step, wherein the pull-down step comprises isolating the first solid phase, and components associated therewith, from other components in the sample or the pre-cleared sample, and wherein the method does not comprise a second target molecule pull-down step.

In some aspects, provided herein is a method of isolating a composition comprising a target molecule from a sample, the method comprising: (a) admixing a pre-cleared sample with a primary antibody specifically recognizing the target molecule, a secondary antibody comprising an affinity label and specifically recognizing the primary antibody, and a first solid phase comprising a first affinity label complexing agent to form a binding composition, wherein the pre-cleared sample is derived from the sample cleared by a first clearing agent and a second clearing agent, wherein the first clearing agent comprises a second solid phase and an antibody binding agent that binds to a population of antibodies, and wherein the primary antibody belongs to the population of antibodies bound by the antibody binding agent, wherein the second clearing agent comprises a third solid phase and a second affinity label complexing agent that forms a complex with the affinity label, wherein a tandem antibody complex is formed in the binding composition in the presence of the target molecule, wherein the tandem antibody complex comprises the target molecule, the primary antibody, the second antibody, and the first solid phase, wherein the primary antibody binds to the target molecule, wherein the secondary antibody binds to the primary antibody, and wherein the first affinity label complexing agent of the first solid phase forms a complex with the affinity label of the secondary antibody; and (b) isolating the composition comprising the target molecule. In some embodiments, the isolating of the composition comprising the target molecule from the pre-cleared sample does not comprise formation of a second tandem antibody complex comprising the primary antibody, the secondary antibody, and another solid phase.

In some aspects, provided herein is a method of isolating a composition comprising a target molecule from a sample, the method comprising: (a) crosslinking the sample; (b) denaturing the crosslinked sample; (c) obtaining the pre-cleared sample, wherein obtaining the pre-cleared sample comprises admixing the sample with the first clearing agent comprising the antibody binding agent that binds to the population of antibodies, and the second clearing agent comprising the second affinity label complexing agent that forms a complex with the affinity label; (d) admixing the pre-cleared sample with a primary antibody specifically recognizing the target molecule, a secondary antibody comprising an affinity label and specifically recognizing the primary antibody, and a first solid phase comprising a first affinity label complexing agent to form a binding composition, wherein the pre-cleared sample is derived from the sample cleared by a first clearing agent and a second clearing agent, wherein the first clearing agent comprises a second solid phase and an antibody binding agent that binds to a population of antibodies, and wherein the primary antibody belongs to the population of antibodies bound by the antibody binding agent, wherein the second clearing agent comprises a third solid phase and a second affinity label complexing agent that forms a complex with the affinity label, wherein a tandem antibody complex is formed in the binding composition in the presence of the target molecule, wherein the tandem antibody complex comprises the target molecule, the primary antibody, the second antibody, and the first solid phase, wherein the primary antibody binds to the target molecule, wherein the secondary antibody binds to the primary antibody, and wherein the first affinity label complexing agent of the first solid phase forms a complex with the affinity label of the secondary antibody; and (e) isolating the composition comprising the target molecule. In some embodiments, the isolating of the composition comprising the target molecule from the pre-cleared sample does not comprise formation of a second tandem antibody complex comprising the primary antibody, the secondary antibody, and another solid phase.

In some aspects, provided herein is a method of isolating a composition comprising a target molecule from a sample, wherein the target molecule is associated with a condensate, or a component thereof, such as a polypeptide component of a biomolecular condensate. In some embodiments, the target is a polypeptide that is, or can be, associated with a condensate. In some embodiments, the target is a nucleic acid that is, or can be, associated with a condensate. In some embodiments, the condensate is a nuclear, cytoplasmic, plasma membrane associated, or secreted extracellular condensate. In some embodiments, methods described herein are capable of isolating molecules involved in transient interactions of a target molecule within the condensate context. For example, in some embodiments, the method described herein is useful for isolating a target molecule associated with additional molecules, i.e., the target molecule is co-isolated with additional associated molecules.

### III. Compositions, Kits, and Systems

Also provided herein are composition, kits, and systems useful for, or resulting from, the methods described herein. In some aspects, provided herein are components useful for the methods described herein.

In some aspects, provided herein is a composition comprising a target molecule obtained from an isolation method described herein. In some embodiments, the tandem-antibody complex comprises a primary antibody, a secondary antibody, a first solid phase, and a target molecule. In some embodiments, the complex does not comprise a first or a second pre-clearing agent.

Also provided herein are kits, for example comprising one or more antibodies, e.g., any of the primary and secondary antibodies described herein, and instructions for performing the methods provided herein. In some embodiments, the kits further comprise one or more clearing agents, e.g., any of the first and second clearing agents described herein. In some embodiments, the one or more antibodies and/or one or more clearing agents further comprise one or more solid phases, to form a binding composition described herein. In some embodiments, the kits further comprise one or more reagents for performing the methods provided herein (e.g., lysis solution, wash solution, and elution solution). In some embodiments, the kits further comprises one or more regents for sample preparation provided herein, such as crosslinking reagents, denaturing regents, and/or inhibitors. In some embodiments, the composition comprising the target molecule is isolated from the sample and is or is a component of a condensate. The various components of the kit may be present in separate containers or certain compatible components may be precombined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for lysing, denaturing, and/or crosslinking the biological sample. In some embodiments, the kits contain molecules, such as antibodies and affinity agents for IP, such as antibodies for forming a tandem-antibody complex, clearing agents for pre-clearing a sample, and/or solid phases for separating a target molecule from a sample. In some aspects, the kit can also comprise any of the reagents described herein, e.g., lysis solution, wash solution, and elution solution. In some embodiments, the kits optionally contain other components, for example inhibitors, enzymes and reagents, buffers, and reagents for additional assays.

### EXAMPLES

### Example 1

This example demonstrates a method for isolating a composition comprising a target from a sample as taught herein. Also provided is a comparison isolation using a known immunoprecipitation (IP) technique, which highlights the improvement in yield and purity obtained using the isolation methods taught herein.

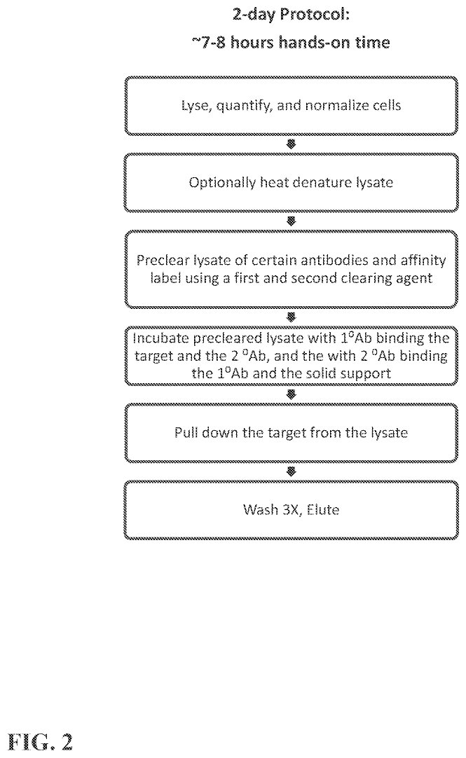

The workflow of the method is outlined in FIG. 2. First, sample cells (HeLa wild type cells) were lysed and protein concentration was normalized. Briefly, the sample cell pellets were collected and transferred into tubes free of DNA, DNase, RNase, and PCR inhibitors (1.5 mL, LoBind® tubes), with about 8×10⁶ cells/pellet. The cell pellets were previously treated with stress, crosslinking, or other desired conditions. About 300 μL ice-cold complete IP lysis buffer (Pierce™ IP Lysis Buffer [25 mM Tris-HCl, pH 7.4, 150 mM NaCl, 1 mM EDTA, 1% NP-40, 5% Glycerol], 2× Halt™ PPI, 10 uM Mg132, 10 mM NEM, DNaseI, 0.1% Tween) was added to each pellet, pellets were resuspended by pipetting, and tubes were rotated end-over-end for 30 minutes (min) at 4° C. Protein concentration was determined by diluting 3 μL of each sample in 12 μL 150 mM/50 mM Tris pH 8.0 (1:5 dilution), and concentration was measured using the Pierce™ 660 nm BCA Assay against a BSA standard. The samples were normalized to ˜750 μg input, and complete IP lysis buffer was added to bring the total volume of each sample to 300 μL.

A pre-clearing step was the performed. Briefly, the pre-clearing step was a two-part pre-clearing comprising a first clearing agent (e.g., Pierce™ Protein A/G magnetic beads) and a second clearing agent (e.g., Pierce™ Streptavidin magnetic beads). The Pierce™ Protein A/G magnetic beads and Pierce™ Streptavidin magnetic beads were prepared for the pre-clearing step prior to IP. 200 μL/sample of Pierce™ Protein A/G magnetic beads (max 1 mL beads/tube) and 74 μL/sample of Pierce™ Streptavidin magnetic beads (max 1 mL beads/tube), were each added to separate tubes free of DNA, DNase, RNase, and PCR inhibitors (1.5 mL, LoBind® tubes). The tubes were placed on a magnetic stand and the supernatants were removed and discarded. 1 mL of 1×TBST was added to the tubes to wash the beads, the solutions were mixed by pipette, and the tubes were rotated end-over-end for about 5 min. The tubes were placed on a magnetic stand and the supernatants were removed and discarded. The wash step was repeated twice. The beads were resuspended in 1×TBST after the final wash, to the starting volume.

The lysate was pre-cleared twice with a combination of 100 μL washed Pierce™ Protein A/G magnetic beads and 12 μL washed Pierce™ Streptavidin magnetic beads (e.g., the first and second clearing agents). 300 μL of the lysate (e.g., the crosslinked and heat denatured lysate) was transferred to each well of a 96-well plate (StorPlate) containing the combination of the washed beads. The lysate was incubated with the combined washed beads in the 96-well plate for 30 min on a hula mixer at 4° C. The plate was placed on a magnetic stand to separate the pre-cleared sample (e.g., supernatant) from the beads (e.g., first and second clearing agents), and the supernatant was transferred to a new 96-well plate (StorPlate) containing the combined washed beads and the lysate was incubated with the combined washed beads for an additional 30 min on a hula mixer at 4° C. The plate was placed on a magnetic stand and the lysate supernatant was transferred to a new 96-well plate (StorPlate) while the bead pellet was discarded, to separate the twice pre-cleared lysate from the beads. Optionally, 10% of the pre-cleared lysate volume could be removed and saved to serve as a lysate control sample in later analyses.

The pre-cleared lysate was incubated with 3 μg the primary antibody (e.g., the anti-bait primary antibody) overnight at 4° C. Subsequently, the lysate was incubated with 6 μg of the biotinylated secondary antibody for 1 hour (h) at 4° C. on a hula mixer. The lysate was then transferred into a 96-well plate (StorPlate) containing 50 μL washed Pierce™ Streptavidin magnetic beads per well, and the plate was placed on a hula mixer at 4° C. for 1 h. The plate was placed on the magnet, and the supernatant comprising the flow through (e.g., the portion of the sample not comprising the target) was carefully removed. The pellet comprising tandem antibody complex (e.g., complex comprising the target molecule, the primary antibody, the secondary antibody, and the Pierce™ Streptavidin magnetic beads) was washed three times with complete ice-cold IP wash buffer (Pierce™ IP Lysis Buffer [25 mM Tris-HCl, pH 7.4, 150 mM NaCl, 1 mM EDTA, 1% NP-40, 5% Glycerol], 1×Halt™ PPI and 0.1% Tween). Specifically, 300 μL complete ice-cold IP wash buffer was added to the beads comprising the tandem antibody complex until the beads were completely resuspended. The plate was placed on the magnet, and the supernatant was carefully removed and discarded (e.g., the flow-through was discarded).

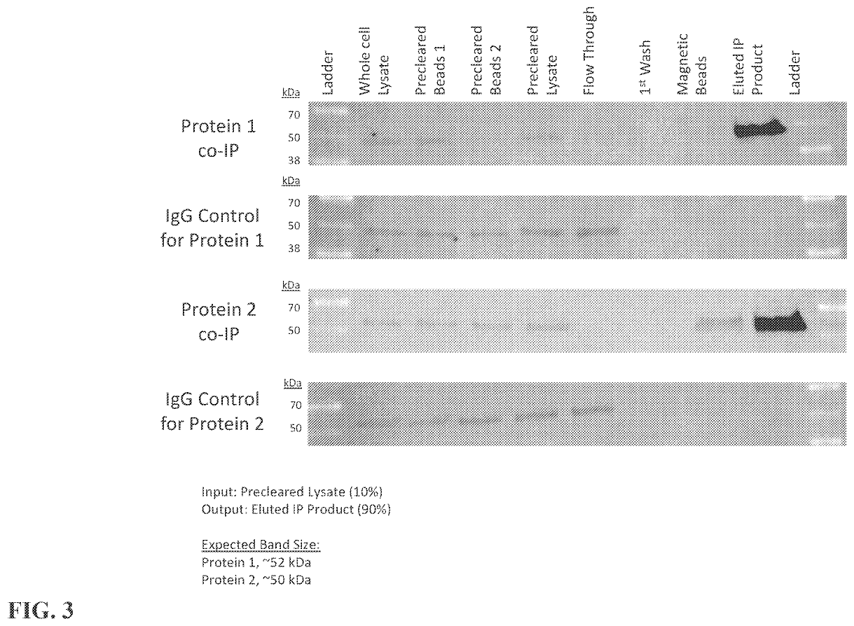

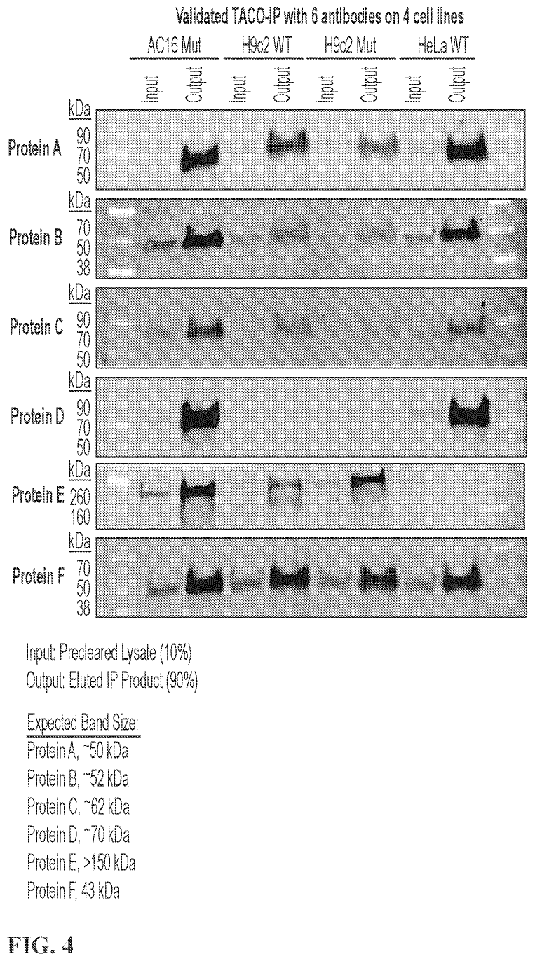

To elute the target molecule from the beads comprising the tandem antibody complex, the beads were resuspended in 55 μL of 2× reducing sample buffer (2× Laemmli buffer with 100 mM TCEP) and transferred to a 96-well plate (LoBind® plate). The samples were heated for 5 min at 95° C., the plate was placed on a magnet, and the supernatant comprising the target molecule was transferred to a new 96-well plate (LoBind® plate). A portion of the sample was frozen −20° C. for mass spectrometry and other downstream analyses. Another portion of the sample was kept at 4° C. for immunoblot analysis, including quality control blots. As shown in FIG. 3, the target molecules (i.e., Protein 1 and Protein 2) were efficiently purified from the sample, and a ˜80-90% recovery.

Compared to the method of Sciuto et al. (Mol & Cell Prot, 17 (5), 2018; Curr Protoc Mol Biol, 125 (1), 2019), the method described herein has several improvements. In particular, the provided method has 1) a streamlined, universal protocol with decreased (˜7-8 h) experimentation time compared to ˜14-16 h experimentation time; 2) increased efficiency of target recovery (˜80-90% recovery) compared to ˜5-10% recovery; and 3) high-throughput capability. Furthermore, the method described herein are robust and allow for an efficient capability to screen many antibodies and/or cell lines, with a reduced need for optimization. The decreased experimentation time of the current method compared to Sciuto et al. allows for a shorter experimental procedure, and the universal, high-throughput nature of the method makes it desirable for any cell type and sample volume. Furthermore, the drastic increase in target recovery versus the previously published method is unexpected and highlights the benefits of this technique. The findings reported herein are unexpected and provide a significant advancement in the field, including in aspects related to ease of protocol, efficiency of for optimization due to robust performance across multiple cell lines and antibodies.

### Example 2

This example demonstrates the application of the methods described herein across multiple cell lines and multiple protein targets.

First, sample cells were lysed and protein concentration was normalized. Briefly, the sample cell pellets were collected and transferred into tubes free of DNA, DNase, RNase, and PCR inhibitors (5 mL, LoBind® tubes), with about 5×10⁷ cells/pellet. The cell pellets were collected and processed without any additional treatments. About 1.5 mL ice-cold complete IP lysis buffer (Pierce™ IP Lysis Buffer [25 mM Tris-HCl, pH 7.4, 150 mM NaCl, 1 mM EDTA, 1% NP-40, 5% Glycerol], 2× Halt™ PPI, 10 uM Mg132, 10 mM NEM, DNaseI, 0.1% Tween) was added to each pellet, pellets were resuspended by pipetting, and tubes were rotated end-over-end for 30 minutes (min) at 4° C. Protein concentration was determined by diluting 3 μL of each sample in 12 μL 150 mM/50 mM Tris pH 8.0 (1:5 dilution), and concentration was measured using the Pierce™ 660 nm BCA Assay against a BSA standard. The samples were normalized to ˜500 μg input, and complete IP lysis buffer was added to bring the total volume of each sample to 300 μL.

A pre-clearing step was then performed. Briefly, the pre-clearing step was a two-part pre-clearing comprising a first clearing agent (e.g., Pierce™ Protein A/G magnetic beads) and a second clearing agent (e.g., Pierce™ Streptavidin magnetic beads). The Pierce™ Protein A/G magnetic beads and Pierce™ Streptavidin magnetic beads were prepared for the pre-clearing step prior to IP. 100 μL/sample of Pierce™ Protein A/G magnetic beads (max 1 mL beads/tube) and 62 μL/sample of Pierce™ Streptavidin magnetic beads (max 1 mL beads/tube), were each added to separate tubes free of DNA, DNase, RNase, and PCR inhibitors (1.5 mL, LoBind® tubes). The tubes were placed on a magnetic stand and the supernatants were removed and discarded. 1 mL of 1×TBST was added to the tubes to wash the beads, the solutions were mixed by pipette, and the tubes were rotated end-over-end for about 5 min. The tubes were placed on a magnetic stand and the supernatants were removed and discarded. The wash step was repeated twice. The beads were resuspended in 1×TBST after the final wash, to the starting volume.

The lysate was pre-cleared once with a combination of 100 μL washed Pierce™ Protein A/G magnetic beads and 12 μL washed Pierce™ Streptavidin magnetic beads (e.g., the first and second clearing agents). 300 μL of the lysate (e.g., the crosslinked and heat denatured lysate) was transferred to each well of a 96-well plate (StorPlate) containing the combination of the washed beads. The lysate was incubated with the combined washed beads in the 96-well plate for 30 min on a hula mixer at 4° C. The plate was placed on a magnetic stand to separate the pre-cleared sample (e.g., supernatant) from the beads (e.g., first and second clearing agents), and the supernatant was transferred to a new 96-well plate (StorPlate) while the bead pellet was discarded, to separate the once pre-cleared lysate from the beads. Optionally, 10% of the pre-cleared lysate volume could be removed and saved to serve as a lysate control sample in later analyses.

The pre-cleared lysate was incubated with 3 μg the primary antibody (e.g., the anti-bait primary antibody) for two hours at 4° C. while on a hula mixer. Subsequently, the lysate was incubated with 6 μg of the biotinylated secondary antibody for 1 hour (h) at 4° C. on a hula mixer. The lysate was then transferred into a 96-well plate (StorPlate) containing 50 μL washed Pierce™ Streptavidin magnetic beads per well, and the plate was placed on a hula mixer at 4° C. for 1 h. The plate was placed on the magnet, and the supernatant was carefully removed (e.g., the flow through was obtained). The tandem antibody complex (e.g., complex comprising the target molecule, the primary antibody, the secondary antibody, and the Pierce™ Streptavidin magnetic beads) was washed three times with complete ice-cold IP wash buffer (Pierce™ IP Lysis Buffer [25 mM Tris-HCl, pH 7.4, 150 mM NaCl, 1 mM EDTA, 1% NP-40, 5% Glycerol], 1× Halt™ PPI and 0.1% Tween). Specifically, 300 μL complete ice-cold IP wash buffer was added to the beads comprising the tandem antibody complex until the beads were completely resuspended. The plate was placed on the magnet, and the supernatant was carefully removed and discarded (e.g., the flow-through was discarded).

To elute the target molecule from the beads comprising the tandem antibody complex, the beads were resuspended in 55 μL of 2× reducing sample buffer (2× Laemmli buffer with 100 mM TCEP) and transferred to a 96-well plate (LoBind® plate). The samples were heated for 5 min at 95° C., the plate was placed on a magnet, and the supernatant comprising the target molecule was transferred to a new 96-well plate (LoBind® plate). Samples were stored at 4° C. for downstream analyses including quality control blots.

As shown in FIG. 4, target molecules were efficiently purified from samples taken from all four cell lines tested (AC16 mutant (Mut), H9c2 wildtype (WT) WT, H9c2 mutant (Mut), and HeLa wild type (WT)). One target molecule, Protein E, served as a negative control for HeLa WT cells, and was efficiently purified from the cell lines in which it was present (AC16 Mut, H9c2 WT, and H9c2 Mut). Proteins A-C and F were efficiently purified from all cell lines. Finally, Protein D was purified from two of the four cell lines (AC16 Mut and HeLa WT). The antibody used was raised against an internal region of Protein D of human origin (e.g., AC16 Mut and HeLa WT, in which Protein D was recognized), and at this time it is unknown if the antibody specifically binds to rat Protein D in rat cell lines (e.g., H9c2 WT and H9c2 Mut).

### Example 3

This example demonstrates the methodology described herein to capture and identify protein interactors to two different bait proteins (bait protein A and bait protein B) within a condensate of interest. These bait proteins were previously confirmed to partition inside a condensate of interest. Using the methodology taught herein, this examples demonstrates the ability of the methodology to use these bait proteins as pull down handles to capture and identify bait protein interactors enriched inside the condensate, which can be very weak and/or transient interactors making such proteins difficult to capture and analyze.

U2OS cells were grown in T175 flasks to obtain at least 0.3 mg/mL of total lysate which is approximately 2.3×10⁷ cells/mL. Samples were grown in biological triplicates using different cell passages, subjected to a condition (including a technical reference condition), crosslinked in situ by addition of dithiobis(succinimidyl propionate) directly to the cells at a final concentration of 0.5 mM, and then allowed to incubate for 30 minutes at room temperature. Sample were then quenched by tris-HCl buffer to prevent further crosslinking and the cells then washed in phosphate buffered saline three times before harvesting them with a scrapper. Pelleted cells were then lysed in RIPA lysis buffer and subsequently used for immunoprecipitation against the desired bait protein following the methodology described in Examples 1 and 2. Conditions evaluated in this experiment included samples with and without daunorubicin (a condensate forming condition), samples with and without the crosslinker used to capture weak/transient protein-protein interactions, samples treated only with IgG beads, and samples with beads only. Technical controls were used to account for spurious proteins that bind non-specifically to the antibodies or beads.

Prior to a quantitative mass spectrometry analysis, captured polypeptides were eluted using heat and a sample buffer containing a reducing agent (TCEP). Quantitative mass spectrometry was performed using multiplexed-MS for 18-plex batches. Because the total number of samples was greater than 18, 17 samples were used in one batch of tandem mass tag labeling and one sample as a bridge sample to compare against other 18-plex batches. This allowed for inter-sample normalization of peptide intensities.

From the output of proteins and their respective peptide intensities, quality control and performed batch correction, contaminant removal, and normalization was performed. Differential analysis was performed between the samples to obtain protein interactors enriched in the condensate-inducing condition versus the non condensate-inducing condition. Furthermore, for both conditions, crosslinker was either added or not added.

A total of 954 prey proteins were identified, from which 98 were significantly enriched in a compound dependent manner and captured in the presence of crosslinker. These proteins were selected for subsequent determination and validation of localization inside the condensate of interest by performing immunofluorescence (IF) assays to assess co-localization with the two bait proteins. Co-localization was used as a metric of partitioning inside the condensate hence validating the candidate interactor as part of the condensate proteome.

From the IF assays performed on the 98 candidate proteins, it was found that 13 proteins (˜8%) co-localized with the two bait proteins suggesting partitioning inside the condensate (fluorescence microscopy images from the IF assays showing co-localization are not provided). The identification of these 13 proteins demonstrated that the methodology described herein enables discovery of interactors (including weak and/or transient interactors) in a condensate of interest.

### Example 4

This examples demonstrates the methodology described herein performed with varying amounts of crosslinker. Protein-protein interactions that occur inside of condensates are typically comprised of weak and/or transient interactions and this example explores more effective capturing of such interactions by using a chemical crosslinker in conjunction with the immunoprecipitation methodology described herein.

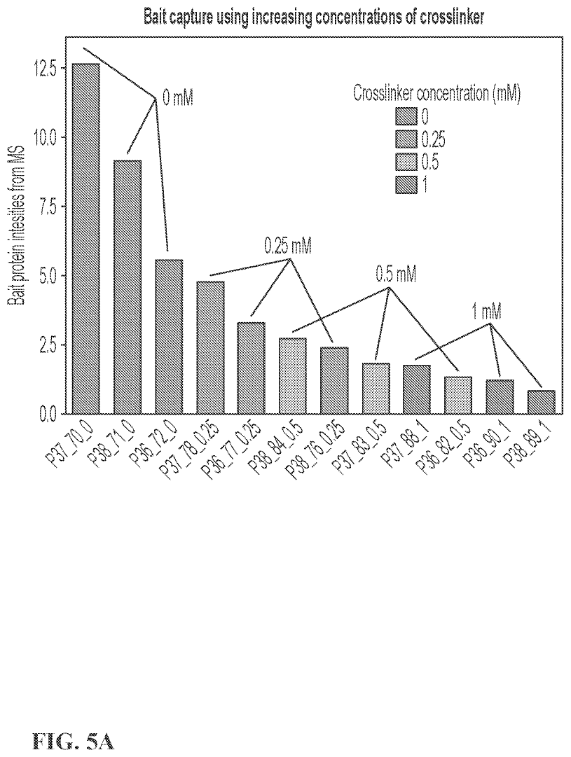

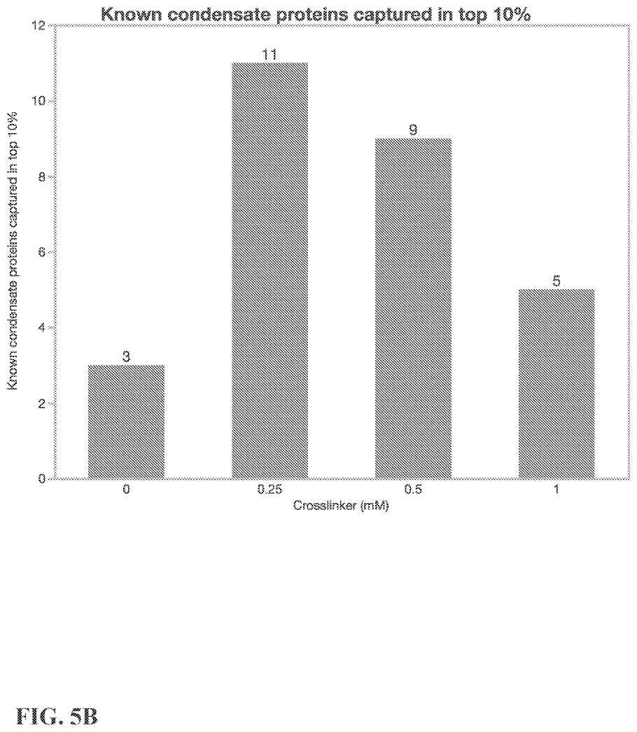

To better understand the effect of crosslinking on the performance of the methodology described herein, pull-downs using a single antibody against one bait protein residing inside a condensate of interest was performed. Specifically, pull-downs were performed in the presence of varying concentrations of dithiobis(succinimidyl propionate) (DSP) crosslinker using the methodology described in Example 3 with 0.25 mM, 0.5 mM, or 1 mM DSP. After performing the pull-downs, samples were analyzed by mass spectrometry for relative quantification. FIG. 5A illustrates intensities of the bait protein across all different samples and replicates. These results show a general trend that an increase in DSP crosslinker concentration results in a decrease in amount of bait protein being captured and analyzed via the mass spectrometry method. At the same time, as shown in FIG. 5B, use of a crosslinker enriched for condensate proteins as compared to a no crosslinker condition based on the number of known condensate proteins identified in the top 10% of hits from the mass spectrometry analysis based on abundance. There was a shift of known condensate proteins towards greater intensity values (greater enrichment) when using DSP crosslinker at a lower concentration of 0.25 mM. For this experiment, use of higher DSP concentrations of 1 mM did not lead to improved enrichment of condensate proteins. This suggests that lower concentrations of crosslinker, such as 0.25 mM DSP, were sufficient to improve capture of the condensate proteome using the methodology taught herein.

1. A method of isolating a composition comprising a target molecule from a sample, the method comprising:
(a) admixing the sample with a primary antibody specifically recognizing the target molecule, a secondary antibody comprising an affinity label and specifically recognizing the primary antibody, and a first solid phase comprising a first affinity label complexing agent to form a binding composition,
wherein a tandem antibody complex is formed in the binding composition in the presence of the target molecule,
wherein the tandem antibody complex comprises the target, the primary antibody, the second antibody, and the first solid phase,
wherein the primary antibody binds to the target molecule,
wherein the secondary antibody binds to the primary antibody, and
wherein the first affinity label complexing agent of the first solid phase forms a complex with the affinity label of the secondary antibody; and
(b) isolating the composition comprising the target molecule,
wherein isolating the composition comprising the target molecule does not comprise formation of a second tandem antibody complex comprising the primary antibody, the secondary antibody, and a solid phase.

2. The method of claim 1, wherein the sample is a pre-cleared sample, and the method further comprises obtaining the pre-cleared sample by admixing the sample with a first clearing agent and a second clearing agent prior to step (a) to form the pre-cleared sample,
wherein the first clearing agent comprises a second solid phase and an antibody binding agent that binds to a population of antibodies, and wherein the population of antibodies comprises the primary antibody specifically recognizing the target molecule,
wherein the second clearing agent comprises a third solid phase and a second affinity label complexing agent that forms a complex with the affinity label in a free state.

3. The method of claim 1, wherein the isolating step comprises a target molecule pull-down step,
wherein the pull-down step comprises isolating the first solid phase, and components associated therewith, from other components in the sample,
wherein the method does not comprise a second target molecule pull-down step.

4. The method of claim 3, wherein isolating the first solid phase, and components associated therewith, comprises subjecting the admixed sample to a force to pellet the first solid phase, and components associated therewith.

5. The method of claim 1, wherein the isolation further comprises a step of removing the target molecule from the first solid phase.

6. A method of isolating a composition comprising a target molecule from a sample, the method comprising:
(a) admixing a pre-cleared sample with a primary antibody specifically recognizing the target molecule, a secondary antibody comprising an affinity label and specifically recognizing the primary antibody, and a first solid phase comprising a first affinity label complexing agent to form a binding composition,
wherein the pre-cleared sample is derived from the sample cleared by a first clearing agent and a second clearing agent,
wherein the first clearing agent comprises a second solid phase and an antibody binding agent that binds to a population of antibodies, and wherein the primary antibody belongs to the population of antibodies bound by the antibody binding agent,
wherein the second clearing agent comprises a third solid phase and a second affinity label complexing agent that forms a complex with the affinity label,
wherein a tandem antibody complex is formed in the binding composition in the presence of the target molecule,
wherein the tandem antibody complex comprises the target molecule, the primary antibody, the second antibody, and the first solid phase,
wherein the primary antibody binds to the target molecule,
wherein the secondary antibody binds to the primary antibody, and
wherein the first affinity label complexing agent of the first solid phase forms a complex with the affinity label of the secondary antibody; and
(b) isolating the composition comprising the target molecule.

7.-15. (canceled)

16. The method of claim 2, wherein the antibody binding agent of the first clearing agent comprises protein G, protein A, protein A/G, protein L, or jacalin.

17. The method of claim 2, wherein the second affinity label complexing agent of the second clearing agent comprises streptavidin, an anti-biotin antibody, or NeutrAvidin.

18.-25. (canceled)

26. The method of claim 1, wherein the secondary antibody binds to the Fc region of the primary antibody.

27. (canceled)

28. The method of claim 1, wherein the affinity label of the secondary antibody comprises biotin, and wherein the first affinity label complexing agent of the first solid phase comprises streptavidin.

29.-31. (canceled)

32. The method of claim 1, wherein the first solid phase comprises a bead.

33.-35. (canceled)

36. The method of claim 2, wherein the first affinity label complexing agent and the second affinity label complexing agent are the same.

37. (canceled)

38. The method of claim 1, further comprising a washing step.

39.-43. (canceled)

44. The method of claim 1, further comprising eluting the target molecule from the first solid phase using an elution solution.

45.-46. (canceled)

47. The method of claim 44, wherein the target molecule is obtained with about 75-95% recovery after eluting.

48.-51. (canceled)

52. A method of isolating a composition comprising a target molecule from a sample, the method comprising:
(a) admixing the sample with a first clearing agent and a second clearing agent to form a pre-cleared sample,
wherein the first clearing agent comprises a second solid phase and an antibody binding agent that binds an antibody species, wherein the antibody species bound by the antibody binding agent is the same as the species of a primary antibody that binds to the target molecule, and
wherein the second clearing agent comprises a third solid phase and a second affinity label binding agent that binds an affinity label;
(b) admixing the pre-cleared sample with the primary antibody, a secondary antibody comprising the affinity label and specifically recognizing the primary antibody, and a first solid phase comprising a first affinity label binding agent, to form a binding composition,
wherein a tandem antibody complex is formed in the binding composition in the presence of the target molecule,
wherein the tandem antibody complex comprises the target molecule, the primary antibody, the second antibody, and the first solid phase,
wherein the primary antibody binds to the target molecule,
wherein the secondary antibody binds to the primary antibody, and
wherein the first affinity label complexing agent of the first solid phase binds to the affinity label of the secondary antibody;
(c) washing the binding composition comprising the tandem antibody complex with a washing solution; and
(d) eluting the composition comprising the target molecule isolated from the tandem antibody complex, to obtain the composition comprising the target molecule isolated from the sample.

53. (canceled)

54. A composition comprising a target molecule obtained from a composition isolation method of claim 1.

55. The method of claim 1, wherein the target molecule is a component of a condensate.

56. The method of claim 1, further comprising crosslinking the sample prior to performing purification steps.

57.-58. (canceled)

59. A kit, comprising a first clearing agent, a second clearing agent, a primary antibody, a secondary antibody, and a first, second, and third solid phase, wherein:
the first clearing agent comprises the second solid phase and an antibody binding agent that binds an antibody species, and wherein the antibody species bound by the antibody binding agent is the same as the species of the primary antibody,
the second clearing agent comprises the third solid phase and a second affinity label binding agent that binds an affinity label,
the primary antibody binds to a target molecule in a sample,
the secondary antibody binds to the primary antibody, and the secondary antibody comprises the affinity label,
the first solid phase comprises a first affinity label complexing agent that binds to the affinity label of the secondary antibody, forming a binding composition comprising a tandem antibody complex in the presence of the target molecule.

60.-61. (canceled)
